(19) United States
(12) Patent Application Publication
(10) Pub. No.: US 2021/0220430 A1
(43) Pub. Date: Jul. 22, 2021

(54) USE OF NOD2 AGONIST FOR THE TREATMENT, PROPHYLAXIS AND/OR DELAY OF THE ONSET OF MULTIPLE SCLEROSIS AND ALZHEIMER?S DISEASE

(71) Applicant: UNIVERSITE LAVAL, Quebec (CA)

(72) Inventors: Serge RIVEST, Québec (CA); Jean GOSSELIN, Québec (CA)

(73) Assignee: UNIVERSITE LAVAL, Quebec, QC (CA)

(21) Appl. No.: 17/056,231

(22) PCT Filed: May 17, 2019
(86) PCT No.: PCT/CA2019/050672

§ 371 Date: Nov. 17, 2020

Related U.S. Application Data
(60) Provisional application No. 62/673,586, filed on May 18, 2018.

(51) Int. Cl. A61K 38/05; A61P 25/28

(52) U.S. Cl. CPC A61K 38/05; A61P 25/28

(57) ABSTRACT

Disclosed is a new treatment for reducing amyloid beta (Aβ), for treating Alzheimer's disease (AD), for improvement of cognitive disorder or learning and memory disorder associated with AD and for the treatment of multiple sclerosis. It was found that a NOD2 agonist could improve phagocytosis of Aβ across the blood brain barrier, to be scavenged by increased concentration of patrolling monocytes caused by the NOD2 agonist, removing Aβ from circulation, thereby preventing its eventual deposit. Also disclosed is a composition for such use.

### FIELD OF THE INVENTION

This invention relates to multiple sclerosis and Alzheimer's disease and its treatments.

### BACKGROUND OF THE INVENTION

Muramyldipeptide (MDP) is derived from minimal bioactive peptidoglycan motif from most Gram-negative and Gram-positive bacteria and is used as adjuvant in different vaccines. MDP is a ligand for intracellular pattern recognition receptor NOD2, which is essential for the innate immune response to MDP.

NOD2 is a member of NLR family of leucine rich repeat proteins. NOD2 receptor is strongly expressed in monocyte precursors that have the ability to differentiate into proinflammatory and patrolling subsets and into macrophages once infiltrating tissues.

In humans, monocyte subsets are characterized by expression levels of CD14 and CD16, as being, classical (CD14⁺⁺CD16⁻), intermediate (CD14⁺⁺CD16⁺) and non-classical (CD14⁺ CD16⁺⁺) subsets. In mice, proinflammatory monocytes are characterized by a combination of cell surface markers (CX3CR1^{low}CCR2⁺Ly6C^{high}), whereas patrolling monocytes are defined as CX3CR1^{high}CCR2⁻Ly6C^{low} cells. Proinflammatory monocytes are involved in inflammatory responses, extravasate in inflamed tissues in a CCR2-dependent manner and thus contribute to local inflammation. On the other hand, patrolling monocytes (also referred to as anti-inflammatory) establish the resident regulatory patrolling monocyte population. Ly6C^{low} monocytes are the population of resident phagocytes that patrol the lumen of blood vessels and enhance tissue repair. In parallel, neurodegenerative diseases, regardless of different etiologies, share common characteristics, such as chronic activation of innate immune cells within the CNS and infiltration of immune cells across blood brain barrier (BBB), especially in multiple sclerosis (MS).

MS is a demyelinating inflammatory disease, which is characterized by T cell-driven autoimmune attack against CNS-derived antigens such as myelin. However, mononuclear phagocytes are the dominant cell type that are abundantly found in active and chronic MS and EAE lesions, and accumulating evidence underline a crucial role of monocytes in MS progression. In particular, recent studies reported that Ly6C^{high} monocytes are the most important cell type in the EAE CNS lesions. The severity of EAE depends on Ly6C^{high} monocytes as they expand exponentially before EAE onset and play crucial roles in the effector phase. In contrast, a recent study demonstrated that non-classical CD14⁺ CD16⁺⁺ monocytes (counterpart to murine patrolling monocytes) are depleted in the circulation of patients with MS. Indeed, the ratio of non-classical CD14⁺ CD16⁺⁺ monocytes to classical (inflammatory) CD14⁺⁺CD16⁻ monocytes was lower in cerebrospinal fluid of patients with MS compared to the control group. Crucial role of monocyte-derived microglia/macrophages in the regulation of neuroinflammation in MS and EAE has also been demonstrated.

Alzheimer's disease (AD) is also characterized by the chronic activation of innate immune cells within the CNS. AD is associated with the accumulation of amyloid beta (Aβ) in the parenchyma and cerebral vasculature due to impaired clearance of the neurotoxic Aβ₁₋₄₀ and Aβ₁₋₄₂ peptides. Several lines of evidence indicate that cerebral amyloid angiopathy (CAA) acts as a significant contributor of the AD pathology. CAA is mainly caused by an impaired Aβ clearance from the cerebral vasculature along perivascular lymphatic drainage pathways. Having more than 90% prevalence in patients with AD, and its relation with cognitive declines clearly show its significant impact on AD pathology. There is a constant equilibrium between Aβ vascular/peripheral and parenchymal levels. Therefore, the clearance of Aβ in perivascular spaces reduces the burden in the parenchyma through equilibrium-driven redistribution.

Thus far, the main pathways of Aβ elimination involve phagocytosis and proteolytic degradation by mononuclear and vascular smooth muscle cells, transcytosis across the blood brain barrier (BBB) and perivascular lymphatic drainage. Therefore, it is now apparent that the neurovascular unit occupies a central position and has a pivotal role for Aβ clearance. The nature of the BBB limits the access to select soluble molecules and circulating leukocytes to the central nervous system (CNS). Among leukocytes, monocytes have a crucial role in AD, as monocyte-derived perivascular macrophages are highly efficient for Aβ phagocytosis.

The use of cholinesterase inhibitors including rivastigmine, donepezil, and memantine, an inhibitor of N-methyl-D-aspartate receptor, are currently the main pharmacologic treatment of Alzheimer's disease (Nygaard H. B., Clin. Ther. 35:1480-1489, 2013; Lannfelt L. et al., J. Int. Med. 275: 284-295, 2014). While those drugs showed effectiveness in reducing dementia symptoms, they cannot stop the progression of the disease. Removal of soluble form of amyloid β peptides thus appeared as an appropriate approach for AD treatment. In this regard, over the past decade, active and passive immunization have been considered for the treatment of this disease. However, results obtained in clinical trials did not provide the expected outcomes.

There are numerous approved disease modifying therapies for MS, such as injectable (Avonex, Copaxone, etc.), oral (Aubagio, Gilenya, etc.) and infused (Lemtrada, Tysabri, etc.) medications. However, the mechanisms underlying their beneficial effects remain unclear and numerous patients do not respond to them while others have very limited responses in relapsing phases of demyelination. They then progress into ongoing paralysis, which inevitably lead to further disability and morbidity. Moreover, there is no medication for patients suffering from primary and secondary progressive MS.

It would be desirable to be provided with a new method for delaying the onset or symptoms of multiple sclerosis and Alzheimer's disease, or for treating multiple sclerosis and Alzheimer's disease, as well as new compositions for such use.

### SUMMARY OF THE INVENTION

In accordance with one aspect of the present invention, there is provided a method for reducing amyloid beta (Aβ) in a patient, said method comprising the step of administering to a patient in need thereof a therapeutically effective dose of a NOD2 agonist.

In another aspect of the invention there is disclosed a method for treating a patient afflicted with Alzheimer's disease (AD) or multiple sclerosis (MS), said method comprising the step of administering to said patient a therapeutically effective dose of a NOD2 agonist.

In a further aspect of the invention, there is disclosed a method for the improvement of cognitive disorder or learning and memory disorder associated with AD, said method comprising the step of administering to a patient in need thereof a therapeutically effective dose of a NOD2 agonist.

Yet in another aspect, there is also disclosed the use of a NOD2 agonist for reducing amyloid beta (Aβ) in a patient.

In a further aspect, there is also disclosed the use of a NOD2 agonist for the treatment of a patient afflicted with Alzheimer's disease (AD) or multiple sclerosis (MS).

Still in a further aspect, there is also disclosed the use of a NOD2 agonist for the improvement of cognitive disorder or learning and memory disorder associated with AD.

In another aspect, there is also disclosed a NOD2 agonist for use in a method as disclosed herein.

In another aspect, there is also disclosed a composition for use in reducing Amyloid beta (Aβ) in a patient, comprising a NOD2 agonist and a pharmaceutically acceptable carrier.

Still in another aspect, there is disclosed a composition for use in treating a patient afflicted with Alzheimer's disease (AD) or multiple sclerosis (MS), comprising a NOD2 agonist and a pharmaceutically acceptable carrier.

In a further aspect, there is also disclosed a composition for use in improving cognitive disorder or learning and memory disorder associated with AD, comprising a NOD2 agonist and a pharmaceutically acceptable carrier.

The present disclosure also provides for the use of the composition as disclosed herein.

In one aspect, reducing the concentration of Aβ means reduction of the concentration of Aβ in circulation. In another aspect, reducing the concentration of Aβ means reducing the quantity of Aβ in the brain. Still in another aspect, reducing the concentration of Aβ means reducing the concentration of Aβ in circulation and reducing the quantity of Aβ in the brain.

### BRIEF DESCRIPTION OF THE DRAWINGS

Embodiments of the present invention will now be described, by way of example only, with reference to the attached Figures, wherein:

FIG. 1 illustrates the flow cytometry gating strategy for monocytes and monocytes sub sets.

FIG. 2 illustrates the flow cytometry gating strategy for T cell subsets.

FIGS. 3A and 3B illustrate the systemic MDP administrations shifting monocyte subsets towards Ly6C^{low} monocytes in the CPZ model. FIG. 3A illustrates the percentage of blood inflammatory Ly6C^{high} monocytes following treatment with vehicle or MDP in normal food, n=5 mice per group, or treated with vehicle or MDP in CPZ-supplemented diet, n=10 mice per group as measured by flow cytometry. Data are expressed as the means±SEM; ***P< or =0.0001 vs. Normal chow-Vehicle, \hat{ }\hat{ }\hat{ }P<0.0001 vs. Cuprizone-supplemented chow-Vehicle. FIG. 3B illustrates the percentage of blood Ly6C^{low} patrolling monocytes following treatment with vehicle or MDP in normal food, n=5 mice per group, or treated with vehicle or MDP in CPZ-supplemented diet, n=10 mice per group) as measured by flow cytometry. Data are expressed as the means±SEM; ***P≤0.0001 vs. Normal chow-MDP, \hat{ }\hat{ }\hat{ }P≤0.0001 vs. Cuprizone-supplemented chow-MDP.

FIGS. 4A-4G illustrate MDP treatment on the modulation of remyelination levels, microglia activation level as well as inflammation in the CNS of cuprizone-fed mice. FIG. 4A illustrates a representation of Black Gold II staining of medial-caudal area of the corpus callosum in saline (top) and MDP (bottom) groups. FIG. 4B illustrates a representation measuring of medial-caudal area of the corpus callosum occupied by myelin in normal chow (vehicle and MDP) and CPZ-supplemented chow (vehicle and MDP) groups. Normal food (vehicle and MDP groups) n=5 mice per group, CPZ-supplemented diet, n=10 mice per group (vehicle and MDP). Data are expressed as the means±SEM; ****P≤0.0001 vs. Normal chow-Vehicle, ^{####}P<0.0001 vs. Normal-chow-MDP. FIG. 4C illustrates Iba1 immunostained on medial-caudal area of the corpus callosum from CPZ-vehicle and CPZ-MDP mice. The area covered by Iba1⁺ staining was measured using a stereological procedure. FIG. 4D illustrates the TLR2 mRNA hybridization signal in the medial-caudal area of the corpus callosum from CPZ-vehicle and CPZ-MDP mice. FIG. 4E illustrates an in situ hybridization signal of trem2 mRNA in medial-caudal area of the corpus callosum from CPZ-vehicle and CPZ-MDP mice. FIG. 4F illustrates a representation of the number of Olig2-immunoreactive staining (olig2⁺ cell/μm³) in medial-caudal area of the corpus callosum from CPZ-vehicle and CPZ-MDP mice. FIG. 4G illustrates the platelet-derived growth factor receptor α (PDGFR-α) mRNA hybridization signal in medial-caudal area of the corpus callosum of CPZ-vehicle and CPZ-MDP mice.

FIGS. 5A to 5I illustrate mice resistance to EAE onset via shifting monocyte subsets towards Ly6C^{low} monocytes and regulation in population of T cells subsets in response to the MDP treatment. FIG. 5A illustrates the clinical scores of WT mice treated with vehicle (n=7) or treated with MDP (n=7) were determined daily after immunization. Data are expressed as the means±SEM; *P≤0.02, **P≤0.002 Mann-Whitney; P≤0.0001 linear regression. FIGS. 5B and 5C illustrate the absolute count of blood Ly6C^{high} and Ly6C^{low} monocytes respectively following treatment with vehicle or MDP in EAE mice as measured by flow cytometry one-week post MDP injections (9-days post immunization). Data are expressed as the means±SEM; **P<0.05 vs EAE-Vehicle, ^{###}P<0.0001 vs EAE-MDP. FIGS. 5D, 5E, and 5F illustrate the absolute count of blood CD3⁺ T cells, CD4⁺ T cells and CD8⁺ T cells respectively following treatment with vehicle or MDP in EAE mice as measured by flow cytometry one-week post MDP injections (9-days post immunization). FIGS. 5G and 5H illustrate the absolute count of blood Foxp3⁺ CD4⁺ T cells and CD4⁺ IL-17⁺ T cells respectively following treatment with vehicle or MDP in EAE mice as measured by flow cytometry one-week post MDP injections (9-days post immunization). FIG. 5I illustrates the absolute count of blood CD8⁺ IL-17⁺ T cells following treatment with vehicle or MDP in EAE mice as measured by flow cytometry one-week post MDP injections (9-days post immunization).

FIGS. 6A to 6K illustrate MDP modulation of monocyte subsets and infiltrating of Ly6C^{high} Ly6C^{low} monocytes, T cell subsets, Ly6G⁺ cells, and CD19⁺ cells in the CNS before onset of EAE. FIG. 6 A illustrates the clinical scores of WT mice treated with vehicle (n=10) or MDP (n=9) were determined daily after immunization. Data are expressed as the means±SEM; *P≤0.02, Mann-Whitney; P<0.0001 linear regression. FIGS. 6B and 6C illustrate the absolute count of CNS Ly6C^{high} and Ly6C^{low} monocytes respectively following treatment with vehicle or MDP in EAE mice as measured by flow cytometry 12-days post-immunization. Data are expressed as the means±SEM; *P<0.02. FIG. 6D illustrates the absolute count of CNS Ly6G⁺ cells following treatment with vehicle or MDP in EAE mice as measured by flow cytometry 12-days post-immunization. Data are expressed as the means±SEM; *P≤0.02. FIGS. 6E, 6F, and 6G illustrate the absolute count of CNS CD3⁺ T cells, CD4⁺ T cells and CD8⁺ T cells respectively following treatment with vehicle or MDP in EAE mice as measured by flow cytometry 12-days post-immunization. Data are expressed as the means±SEM; *P≤0.04. FIG. 6H illustrates the absolute count of CNS Foxp3⁺ CD4⁺ T cells following treatment with vehicle or MDP in EAE mice as measured by flow cytometry 12-days post-immunization. Data are expressed as the means±SEM; **P≤0.007. FIG. 6I illustrates the absolute count of CNS IL-17⁺ CD4⁺ T cells following treatment with vehicle or MDP in EAE mice as measured by flow cytometry 12-days post-immunization. FIG. 6J illustrates the absolute count of CNS CD19⁺ cells following treatment with vehicle or MDP in EAE mice as measured by flow cytometry 12-days post-immunization. FIG. 6K illustrates the immunoblot analysis of Iba1 protein expression in the CNS showing no significant difference between control (EAE-Vehicle) and treatment (EAE-MDP) groups.

FIGS. 7A to 7E illustrate the critical role of NOD2 receptor in MDP-dependent immune modulation and EAE resistance in mice. FIG. 7A illustrates the clinical scores determined daily after immunization in WT mice treated with MDP (n=6), EAE-NOD2^{-/-} mice treated with vehicle (n=6) and EAE-NOD2^{-/-} mice treated with MDP (n=6). Data are expressed as the means±SEM; *P≤0.01 vs. EAE-MDP, days 15&16, ^{#}P≤0.01 vs. EAE-Vehicle, days 14-16, Mann-Whitney; P≤0.0001 linear regression. FIG. 7B illustrates the absolute count of blood Ly6C^{high} monocytes following treatment with vehicle or MDP in EAE mice and EAE-NOD2^{-/-} mice as measured by flow cytometry 21-days post immunization. FIG. 7C illustrates the absolute count of blood Ly6C^{low} monocytes following treatment with vehicle or MDP in EAE mice and EAE-NOD2^{-/-} mice as measured by flow cytometry 21-days post immunization. Data are expressed as the means±SEM; **P≤0.008 vs. EAE-WT-MDP. FIGS. 7D and 7E illustrate the absolute count of blood Foxp3⁺ CD4⁺ T cells and CD4⁺ IL-17⁺ T cells respectively following treatment with vehicle or MDP in EAE mice and EAE-NOD2^{-/-} mice as measured by flow cytometry 21-days post immunization.

FIGS. 8A to 8C illustrate the results of flow cytometry analysis of blood Ly6C^{high} (FIG. 8A), Ly6C^{inter} (FIG. 8B), and Ly6C^{low} (FIG. 8C) monocytes at 3 and 6 months following MDP or saline treatments (i.p., every 72 hours). ****P≤0.0001 as compared to indicated groups.

FIGS. 9A to 9F illustrate the regulation of monocyte subsets and improvement in memory deficits following chronic MDP administration over 6 months (high frequency) in APP mice. FIG. 9A illustrates the percentage of blood inflammatory Ly6C^{high} monocytes at two time points (3 and 6 months) following chronic MDP administration over 6 months (high frequency) in APP mice. Data are expressed as the means±SEM; ***P≤0.0004 vs. APP-Vehicle in 3 months, ^{###}P≤0.0004 vs. APP-Vehicle in 6 months. FIG. 9B illustrates the percentage of blood Ly6C^{low} patrolling monocytes at two time points (3 and 6 months) following chronic MDP administration over 6 months (high frequency) in APP mice. Data are expressed as the means±SEM; ***P≤0.0004 vs. APP-MDP in 3 months, ^{###}P≤0.0004 vs. APP-MDP in 6 months. FIG. 9C illustrates the total number of errors made on Day 1 (D1), Day 2 (D2), and Day 3 (D3) in APP-MDP and APP-Vehicle groups in learning performance in position habit acquisition at the two time-points (3 and 6 months). FIG. 9D illustrates the total number of errors made on Day 1 (D1), Day 2 (D2), and Day 3 (D3) in APP-MDP and APP-Vehicle groups in learning performance in reversal learning training at the two time points (3 and 6 months). FIG. 9E illustrates the percentage of mice in APP-MDP and APP-Vehicle groups made errorless trials in Day 1 in reversal learning training at the two time-points (3&6 months). FIG. 9F illustrates the average of total errors in APP-MDP and APP-Vehicle groups in learning performance in reversal learning training at the two time points (3 and 6 months).

FIGS. 10A to 10D illustrate the regulation of monocyte subsets and improvement in memory deficits following chronic MDP administration over 3 months (low frequency) in APP mice. FIG. 10A illustrates the absolute count of blood inflammatory Ly6C^{high} monocytes in WT and APP mice and following chronic MDP administration over 3 months (low frequency). Data are expressed as the means±SEM; ^{S}P≤0.01 vs. WT-Vehicle. FIG. 10B illustrates the absolute count of blood Ly6C^{low} monocytes in WT and APP mice and following chronic MDP administration over 3 months (low frequency). Data are expressed as the means±SEM; ^{SS}P≤0.003 vs. WT-MDP, ^{%%}P≤0.007 vs APP-MDP. FIG. 10C illustrates the total number of errors made on Day 1 (D1), Day 2 (D2), and Day 3 (D3) in WT and APP mice in learning performance in position habit acquisition following chronic MDP administration over 3 months (low frequency). Data are expressed as the means±SEM; **P≤0.003 vs. APP-MDP D1, ***P≤0.0004 vs APP-MDP D1. FIG. 10D illustrates the total number of errors made on Day 1 (D1), Day 2 (D2), and Day 3 (D3) in WT and APP mice in learning performance in position habit acquisition following chronic MDP administration over 3 months (low frequency). Data are expressed as the means±SEM; *P≤0.01 vs. APP-MDP D1.

FIGS. 11A to 11N illustrate effect of MDP treatment on microglial activation and Aβ burden in the brain of APP mice. FIG. 11A illustrates the average number of Iba1⁺ associated to 6E10⁺ plaques to hippocampus area (μm²) of APP mice treated with vehicle and MDP. FIG. 11B illustrates the average number of 6E10⁺ plaques to hippocampus area (μm²) of APP mice treated with vehicle and MDP. FIG. 11C illustrates the average number of Iba1⁺ associated to 6E10⁺ plaques to cortex area (μm²) of APP mice treated with vehicle and MDP. FIG. 11D illustrates the average number of 6E10⁺ plaques to cortex area (μm²) of APP mice treated with vehicle and MDP. FIGS. 11E and 11F illustrate the representation of iba1 (red), 6E10 (green) and DAPI (blue)-immunoreactivity in hippocampus of APP mice treated with vehicle (left) and MDP (right) (scale bar, 20 μm). FIGS. 11G and 11H illustrate the representation of 6E10 (red)-immunoreactivity in hippocampus of APP mice treated with vehicle (11G) and MDP (11H) (scale bar, 100 μm). FIG. 11I illustrates the concentrations (picogram/ml) of Aβ 40 and Aβ 42 in the cortex and hippocampus of APP mice treated with vehicle and MDP were quantified by ELISA. FIG. 11J illustrates the Aβ 40 and Aβ 42 ratios in the cortex and hippocampus of APP mice treated with vehicle and MDP, which were quantified by ELISA. FIG. 11K illustrates an immunoblot analysis of APP protein levels in the cortex and hippocampus of APP mice treated with vehicle and MDP. FIG. 11L illustrates an immunoblot analysis of Iba1 protein levels in the cortex and hippocampus of APP mice treated with vehicle and MDP. FIG. 11M illustrates an immunoblot analysis of TREM2 protein levels in the cortex and hippocampus of APP mice treated with vehicle and MDP. FIG. 11N illustrates an immunoblot analysis of COX2 protein levels in the cortex and hippocampus of APP mice treated with vehicle and MDP. Data are expressed as the means±SEM; ***P≤0.0001 vs. APP-MDP.

FIGS. 12A to 12F illustrate the effect of MDP treatment on key proteins involved in synaptic functions, Aβ vascular clearance, and cerebrovascular monocyte adhesion. FIG. 12A illustrates the immunoblot analysis of synaptophysin protein levels in the cortex and hippocampus of APP mice treated with vehicle and MDP, n=10 mice per group. FIG. 12B illustrates the immunoblot analysis of PSD95 protein levels in the cortex and hippocampus in the brain of APP mice treated with vehicle and MDP, n=10 mice per group. Data are expressed as the means±SEM; *P< or =0.03. FIG. 12C illustrates the immunoblot analysis of LRP1 protein levels in the cortex and hippocampus of APP mice treated with vehicle and MDP, n=10 mice per group. Data are expressed as the means±SEM; *P≤0.03. FIG. 12D illustrates the immunoblot analysis of MCP1 protein levels in the cortex and hippocampus of APP mice treated with vehicle and MDP, n=10 mice per group. Data are expressed as the means±SEM; *P≤0.03. FIG. 12E illustrates the immunoblot analysis of VCAM protein levels in the cortex and hippocampus of APP mice treated with vehicle and MDP, n=10 mice per group. FIG. 12F illustrates the immunoblot analysis of ICAM protein levels in the cortex and hippocampus of APP mice treated with vehicle and MDP, n=10 mice per group. Data are expressed as the means±SEM; ****P≤0.0001.

FIGS. 13A to 13I illustrate MDP-mediated shifting Ly6C^{high} towards Ly6C^{low} monocytes selectively attracted to small cerebrovascular containing Aβ aggregates. FIGS. 13A, 13D, and 13G illustrate a representation of a two-photon intravital imaging of cortical blood vessels from 12 months WT (13A) and APP/PS1/CX3CR1^{-/GFP} (13D and 13G) mice. Mouse in FIGS. 13A and 13D received 10 mg/kg i.p. MDP for 4 consecutive days while mouse in FIG. 13G received saline. CX3CR1^{gfp}-expressing cells such as microglia, perivascular macrophages, and monocytes are in green, blood vessels are in gray (Qdot 705), and Aβ in red (Congo red). Scale bar, 50 μm. FIGS. 13B, 13E and 13H illustrate a representation of the flow cytometry analysis of blood monocytes (mono) Ly6C^{low} patrolling (pat), Ly6C^{int} intermediate (int) and Ly6C^{high} inflammatory (inf) cells in WT (13B) and APP/PS1/CX3CR1^{-/GFP} (13E) and (13H). Mouse treated with MDP (13B) and (13E) have higher number of total monocytes compared to saline group (13H). FIGS. 13C, 13F, and 13I illustrate a 5-minute time lapse quantification of CX3CR1^{-/GFP}-expressing cells observed in cortical blood vessels (13A), (13D) and (13G) before treatment (day 0) and 1 week after the first injection (day 7). Despite the same percentage of total monocytes (13B) and (13E), crawling GFP-cells are more frequent in MDP-treated APP/PS1/CX3CR1^{-/GFP} mouse vessels containing small Aβ aggregates than Aβ-free vessels of WT mouse where crawling GFP cells are rarely observed.

FIG. 14 illustrates monocytes being selectively attracted to small Aβ aggregates in response to MDP. Crawling monocytes are recruited in specific small Aβ aggregates (black arrowheads) present on APP/PS1/CX3CR1^{-/GFP} cortical blood vessels (scale bar, 20 μm) following MDP administration.

FIGS. 15A to 15D illustrate Western blot analysis of BACE1 (FIG. 15A) and LRP1 (FIG. 15B) and the related corrected optical densities measured, expressed in fold increase of BACE1 (FIG. 15C) and LRP1 (FIG. 15D) in the brain of APP mice after 6 months of MDP or saline treatment (i.p., every 72 hours). *P≤0.05 as compared to indicated groups.

FIGS. 16A and 16B illustrate Western blot analysis of PSD95 (FIG. 16A) and the related corrected optical densities measured, expressed in fold increase of PSD95 (FIG. 16B) in the brain of APP mice after 6 months of MDP or saline treatment (i.p., every 72 hours). *P<0.05 as compared to indicated groups.

FIGS. 17A to 17D illustrate Western blot analysis of COX2 (FIG. 17A) and MCP1 (FIG. 17B) and the related corrected optical densities measured, expressed in fold increase of COX2 (FIG. 17C) and MCP1 (FIG. 17D) in the brain of APP mice after 6 months of MDP or saline treatment (i.p., every 72 hours). *P≤0.05 and ****P≤0.0001 as compared to indicated groups.

FIGS. 18A and 18B illustrate the acquisition-learning phase (FIG. 18A) and the reversal-learning phase (FIG. 18B) of Water T-maze experiment in WT and APP mice treated for 6 months with MDP or saline (i.p., every 72 hours).

FIGS. 19A and 19B illustrate the learning curve—training (FIG. 19A) and the learning curve—reversal (FIG. 19B) in a Water T-maze experiment in WT and APP mice treated for 3 months with MDP or saline (i.p., 1 time/week).

### DETAILED DESCRIPTION OF THE INVENTION

As used herein, the expression “therapeutically effective amount” refers to an amount of active compound or pharmaceutical agent that elicits the biological or medicinal response in a subject that is being sought by a researcher, veterinarian, medical doctor or other clinician, which may include inter alia an increase in the number of patrolling monocytes in the blood of a subject, and alleviation of the symptoms of the disease or condition being treated. Methods are known in the art for determining therapeutically and prophylactically effective doses for the pharmaceutical formulation as taught herein.

As used herein, “Ly6C^{high} monocytes” is used interchangeably with “Ly6C^{hi} monocytes” and “Inflammatory monocytes”.

As used herein, “patrolling monocytes” is used interchangeably with “Ly6C^{low} monocytes” and “non-classical monocytes”.

As used herein, although MDP has been tested, this compound is a NOD2 agonist. However, any NOD2 agonist having a carboxyl group on the glutamine, instead of an ester as in the case of murabutide, can be used in the present invention. This carboxyl group seems very important for activation of other NOD2 agonists, as described in Girardin et al. (J. Biol. Chem. 278: 41702-41708, 2003). According to one aspect, NOD2 agonist and MDP preferably mean to refer interchangeably to any one of MDP, NAcMDP, N-glycolyl-MDP, L18-MDP, and M-TriLYS.

In the present invention, it was first investigated whether patrolling monocytes could play a role in the clearance of vascular Aβ, using a 2-photon intravital laser-scanner system and observed that Ly6C^{low} monocytes monitor and crawl inside the lumen of blood vessels independently of the blood flow (Michaud J P et al., Cell Rep. 5: 646-653, 2013). Patrolling monocytes display several filopodia-like protrusions in contact with the endothelium as well as large endosomes for containing Aβ and clearing it from the vascular elements.

In this invention, the inventors investigated whether MDP could influence neuropathology of mouse models of MS and AD by regulating monocyte cell subsets. The inventors performed in vivo studies of immunomodulatory effects of MDP in two mouse models of MS (cuprizone and EAE) and also APP_{Swe}/PS1 mice (referred herein to APP mice) mouse model of AD. It was found that MDP administrations in both models of MS convert Ly6C^{high} into Ly6C^{low} monocytes, but there were no significant changes in demyelination levels in the cuprizone model. On the other hand, peripheral MDP administrations in EAE delayed disease onset in a NOD2-dependent manner, decreasing the number of Ly6C^{high} infiltrating the CNS and reducing the number of T cells. Using NOD2^{-/-} mice, it was found that NOD2 receptor plays a critical role in MDP-dependent immune modulation and EAE resistance. MDP administrations in APP mouse model of AD also converted Ly6C^{high} into Ly6C^{low} monocytes, which was associated with improvement in memory deficits together with the increase expression of markers of synaptic plasticity and Aβ clearance. Finally, two-photon intravital microscopy showed that Ly6C^{low} monocytes are more recruited to the brain vasculature and are able to phagocyte Aβ peptides in APP mice following MDP administrations.

Evidence was also provided that Ly6C^{low} patrolling monocytes are located at a key position, contacting frequently and selectively Aβ-laden veins, and scavenging Aβ from the lumen. Over the course of the disease, such natural interactions could be less effective and contribute to the marked vascular Aβ deposition. Fiala et al. (J Alzheimers Dis. 7: 221-232, 2005) reported that monocytes isolated from AD patients and exposed to Aβ exhibited low phagocytosis, abnormal cytokines release and increased apoptosis. It was then postulated that stimulating the production of new and functional blood monocytes could counteract these defects. Since an equilibrium exists between parenchymal, vascular, and peripheral Aβ levels, increasing vascular Aβ clearance by patrolling monocytes could have significant impact on AD. In this regard, reducing the migration, phagocytosis, or number of mononuclear cells in transgenic AD mice is detrimental while compounds increasing their number and phagocytic activity could be beneficial.

NOD2 is a member of the nucleotide-binding oligomerization domain-(NOD)-like receptor (NLR) family. While NOD2 was initially believed to be solely involved in the recognition of bacterial motifs, it is now recognized that NOD2 can also sense RNA viruses. NOD2 is expressed in cells of both myeloid and lymphoid origins like macrophages, monocytes, astrocytes, microglia, endothelial cells and T lymphocytes. NOD2 is also suspected to contribute to regulate inflammation and to maintain tissue homeostasis, since NOD2 variants are associated with inflammatory diseases such as Crohn's disease, Blau syndrome, and early onset sarcoidosis.

Triggering of NOD2 by peptidoglycan ligands leads to the recruitment of the signaling element RIP2 and to the activation of NF-κB and MAP kinase, resulting in the production of inflammatory cytokines and chemokines. On the other hand, when NOD2 recognizes viral ssRNA, an antiviral response is activated via the recruitment of the IPS-1 adaptor molecule followed by the activation of IRF3 and IRF7 and the production of type 1 IFN. Production of such inflammatory mediators contributes to recruit and activate immune cells including neutrophils and monocytes. Many NOD2 agonists are known in the art (Fritz J. H. et al., Nature Immunol 7: 1250-1257, 2006; Fritz J. H. et al., Eur. J. Immunol. 35: 2459-2470, 2005). For example, the minimal molecular bacterial motif detected by NOD2 is the muramyl dipeptide MurNAc-L-Ala-D-isoGln (MDP) (Girardin S. E. et al., J. Biol. Chem. 278: 8869-8872, 2003; Inohara N. et al., J. Biol. Chem. 278: 5509-5512, 2013). Upon MDP sensing, MAP kinases and transcription factors NF-kB and IRFS are activated. The N-glycolyl MDP is a more potent agonist of NOD2 than the classical N-acetyl MDP at stimulating inflammatory genes. NOD2 can also detect the peptidoglycan structure MurNAc-L-Ala-D-Glu-L-Lys (MtriLys) (Fritz J. H. et al., Eur. J. Immunol. 35: 2459-2470, 2005). The synthetic NOD2 agonist, N-Acetyl-muramyl-Ala-D-isoglutaminyl-Ns-steroyl-Lys (MDP-Lys or L18) can mimic bacterial peptidoglycan to act as an adjuvant in cell-mediated immunity (Fujimura T. et al., J. Dermatol. 62: 107-115, 2011). Murabutide is another synthetic derivative from MDP that may act as an immumodulator to potentiate the immune response (Feinen B. et al., Clin. Vaccine Immunol. 21: 580-586, 2014).

While several studies have clearly recognized NOD2 as a key receptor in innate immune defense against microbial infection and to play a potential role in inflammatory diseases, it is still unknown whether NOD2 can activate cellular signals that are involved in the regulation of homeostasis. In this regard, it was recently reported that in vivo administration of MDP to mice leads to the emergence of blood patrolling monocytes expressing similar phenotype and functions of anti-inflammatory Ly6C^{low} monocytes (Lessard A. J. et al., Cell Rep. 20: 1830-1843, 2017), suggesting that these converted monocytes could contribute to regulate the inflammatory response to maintain homeostasis. Current available treatments for Alzheimer's disease are limited to reduce dementia symptoms and do not delay or arrest progression of the disease. Furthermore, during the last decade, targeting amyloid-β peptides have been considered as potential therapeutic approach for the treatment of Alzheimer's disease. Such approaches, however, have not yet yielded to conclusive results. Moreover, it is now reported herein that patrolling monocytes could have a significant impact in amyloid-β clearance, providing for a method to increase levels of patrolling monocytes, thereby providing for a novel and attractive therapeutic approach for the treatment of Alzheimer's disease, which has now been investigated herein.

In the present invention, it is demonstrated herein selective immunomodulatory and therapeutic effects of MDP on mouse models of MS and AD. The inventors found that MDP administrations in experimental autoimmune encephalomyelitis (EAE) mouse model of MS delayed onset of disease, improved clinical scores, and reduced number of Ly6C^{high} cells that infiltrated into the CNS. In addition, MDP treatment regulated multiple effector T cell subsets. The results also demonstrated that NOD2 receptor plays a critical role in MDP-mediated EAE resistance. In parallel, it was also noted that MDP injections improved cognitive declines in APP_{Swe}/PS1 mouse model of AD and increased expression levels of PSD95 and LRP1, which are involved in synaptic plasticity and Aβ elimination, respectively. Using intravital two-photon microscopy, it was observed that Ly6C^{low} monocytes are actively recruited to the brain vasculature and are able to pick up Aβ peptides in APP_{Swe}/PS1 mice following MDP treatment. The results demonstrate that MDP is beneficial in both the early and progressive phases of MS as well as early phase and to some extent later phases of AD.

MDP administrations regulate monocyte subsets mainly by converting Ly6C^{high} into Ly6C^{low} monocytes. Critical roles of monocytes in MS and AD pathologies make them important potential therapeutic targets. Here, the inventors performed in vivo studies of immunomodulatory effects of MDP in two mouse models of MS (cuprizone and EAE) and also APP mouse model of AD. The inventors have shown that MDP shifts Ly6C^{high} towards Ly6C^{low} monocytes in both cuprizone and EAE mouse models of MS. Although demyelination levels did not change in the cuprizone model, the results obtained from the EAE model were promising. In fact, MDP treatments delayed disease onset, which was accompanied by a significant reduction in number of Ly6C^{high} cells in blood and into the CNS. Interestingly, the number of some T cell subsets was also affected by the MDP treatment. The inventors next determined whether NOD2 receptor is involved in MDP-mediated therapeutic effects and it was discovered that NOD2 receptor plays a critical role in MDP-mediated EAE resistance. The same immunomodulatory effect of MDP on monocyte subsets in terms of converting Ly6C^{high} to Ly6C^{low} monocytes in APP mouse model of AD was also observed. In addition, MDP treatments in APP mice significantly increased expression (protein) levels of PSD95, LRP1, and COX2, together with a decrease in ICAM-1. The inventors then performed intravital two-photon microscopy and observed that Ly6C^{low} monocytes were actively recruited to the brain vasculature and were able to pick up Aβ peptides in response to the MDP treatment.

It is shown herein that MDP-treated mice were highly resistant to EAE, which is mediated by regulation of monocyte subsets and to some extent T cell subsets. Indeed, clinical scores confirmed that MDP-treated mice were more protected from disease progression, delayed significantly disease onset, and decreased incidence of disease. These observations were correlated with significant reduction and increase in number of Ly6C^{high} and Ly6C^{low} monocytes, respectively, both in the circulation and CNS. Several previous studies have unraveled crucial roles of monocyte subsets and monocyte-derived macrophages in EAE and MS. In particular, rapid influx of Ly6C^{high} monocytes from the circulation or peripheral reservoirs resulting in onset of EAE, as CCR2-deficient mice are resistant to EAE (Fife B. T. et al., J Exp. Med. 192: 899-906, 2000; Izikson L. et al., Clin. Immunol. 103: 125-131, 2002). Furthermore, numbers of Ly6C^{high} monocytes increase in the blood within 1 day after immunization in EAE mice (Mishra M. K. et al., Am. J. Pathol. 181: 642-651, 2012). Additionally, administration of dipyridamole, a medication used clinically for secondary prevention in stroke showed inhibitory effects on activation of proinflammatory myeloid cells (Sloka S. et al., J. Neuroinflam 10: 855, 2013). Significance of MDP-mediated reduction in Ly6C^{high} monocytes is not limited to production of proinflammatory cytokines and chemokines (King I. L. et al., Blood 113: 3190-3197, 2009). It is also related to their effects on antigen presentation that activates T cells (Benveniste E. N., J. Mol. Med. 75: 165-173, 1997) and generation of oxidative stress and other mediators of injury (Nikić I. et al., Nat. Med. 17: 495-499, 2011; Van Horssen J. et al., Biochim. Biophys. Acta 1812: 141-150, 2011; Yamasaki R. et al., J. Exp. Med. 211: 1533-1549, 2014; Mossakowski, A. A. et al., Acta Neuropathol 130: 799-814, 2015).

In parallel, the results obtained indicated a significant increase in Ly6C^{low} monocyte population in peripheral circulation as well as CNS.

Modulation of monocyte subsets can modify population of monocyte-derived macrophages in systemic organs as well as in the CNS. For example, Ly6C^{low} monocytes can include perivascular macrophages (Sorokin L., Nat. Rev. Immunol. 10: 712-723, 2010; Agrawal S. M. et al., Brain 136: 1760-1777, 2013). Some studies reported that depletion of both perivascular and meningeal macrophages curtails EAE severity (Greter M. et al., Nat. Med. 11: 328-334, 2005). In parallel, immune cell activation and infiltration have been shown in the choroid plexus of MS patients (Engelhardt B. et al., Microsc. Res. Tech. 52: 112-129, 2001; Vercellino M. et al., J. Neuroimmunol. 199: 133-141, 2008) and EAE animals (Brown D. A. and P. E. Sawchenko, J. Comp. Neurol. 502: 236-260, 2007). In addition, choroid plexus macrophages-mediated inflammation in cerebrospinal fluid may directly impact meningeal and perivascular inflammation (Vernet-der Garabedian, Lemaigre-Dubreuil et al. 2000; Bragg D. et al., Neurobiol. Dis. 9: 173-186, 2002, Bragg D. et al., J. Neurovirol. 8: 225-239, 2002).

In addition, further analysis of the results obtained demonstrated strong tendency (P=0.0591) for decrease in CD3⁺ as well as CD4⁺ T cells (P=0.0553) in the circulation. More importantly, MDP treatments attenuated significantly the influx of T cell subsets including CD3⁺, CD4⁺ and CD8⁺ T cells into the CNS. It is possible to consider that this phenomenon is mediated by the regulatory effects of MDP on monocyte subsets, as previous reports showed that monocyte/macrophage regulation has the ability to change T cell subsets infiltration (Bauer J. et al., Glia 15: 437-446, 1995; Tran E. H. et al., J. Immunol. 161: 3767-3775, 1998). The results demonstrated that MDP treatments reduce CD4⁺CD25⁺Foxp3⁺ regulatory T (Tregs) cell numbers in both the circulation and the CNS. The increase in non-suppressing Tregs cells together with pro-inflammatory T cells at peak of EAE disease in the CNS and in the synovium of rheumatoid arthritis models have been reported (Cao D. et al., Eur. J. Immunol. 33: 215-223, 2003; O'Connor R. A. et al., J. Immunol. 179: 958-966, 2007). The data obtained here suggest that MDP-mediated decrease in Tregs may be another factor modulating neuroinflammation in EAE mice. IL-17⁺CD4⁺ T cells is another T cell subset that plays a key role in the MS disease, especially its role in CNS autoimmunity (Luger D. et al., J. Exp. Med. 205: 799-810, 2008; Lee S. Y. and J. M. Goverman, J. Immunol. 190: 4991-4999, 2013). The inventors identified a tendency (P=0.0879) in a decreased number of IL-17⁺CD4⁺ T cells in both the circulation and the CNS (P=0.0619) of EAE mice treated with MDP. Among many roles in triggering autoimmunity, IL-17⁺CD4⁺ T cells play a crucial role in the BBB breakdown (Huppert J. et al., FASEB J. 24: 1023-1034, 2010). Such a decrease in T cell subsets including IL-17⁺CD4⁺ T cells could be mediated by anti-inflammatory monocytes, which are known to promote apoptosis of T lymphocytes (Moline-Velazquez V. et al., Brain Pathol. 21: 678-691, 2011).

Also demonstrated herein is the critical role of NOD2 in MDP-mediated immunomodulatory and EAE resistance. Indeed, EAE-NOD2^{-/-}-MDP mice showed higher disease incidence, slightly earlier of disease, and slightly higher disease severity and hind-limb paralysis when compared with EAE-WT-MDP group. More importantly, MDP treatment did not regulate monocyte subsets in EAE-NOD2^{-/-} mice compared to EAE-WT mice. The inventors did not observe regulation of CD3⁺, CD4⁺, and CD8⁺ T cell subsets in NOD2^{-/-} mice treated with MDP. As previously reported, these results provide evidence that the effects of MDP on immune cells depend on NOD2 receptor.

The critical role of Ly6C^{low} monocytes in Aβ clearance via internalization of Aβ and efficiently eliminate Aβ microaggregates had been previously reported (Michaud J.-P. et al., Proc. Natl. Acad. Sci. USA 110: 1941-1946, 2013). Consequently, the inventors examined the potential therapeutic effect of MDP in APP mouse model of AD in two protocols. In both of them, MDP shifted monocyte subsets and improved cognitive deficits as demonstrated by behavioral tests. More importantly, the results indicated that chronic administration of MDP at lower frequency is sufficient to delay the appearance of an Alzheimer-like phenotype. Given that mice treated with MDP showed improvement in memory deficits, the inventors first examined microglial activation and Aβ levels. However, no changes in Aβ burden or microglial activation were observed, suggesting that memory/learning improvements observed in behavioral tests are dependent on other factor(s).

Previous reports demonstrated that the degree of synapse loss is a stronger correlate of cognitive decline in AD than counts and/or size of plaques (DeKosky S. T. and S. W. Scheff, Ann. Neurol. 27: 457-464, 1990; Terry R. D. et al., Ann. Neurol. 30: 572-580, 1991; Hong S. et al., Science 352: 712-716, 2016). The inventors found that PSD95 protein expression level significantly increased in APP-MDP compared to that of control. PSD95 is the most abundant protein in the excitatory postsynaptic density. Furthermore, PSD95 is a master regulator of neuronal plasticity and memory (Bustos F. J. et al., Brain 140: 3252-3268, 2017) and has previously been showed to be decreased in APP mouse model of AD (Hou Y et al., Neuropharmacology 58: 911-920, 2010). Interestingly, other studies demonstrated a role of PSD95 in interacting and regulating adhesion molecules, signaling proteins, scaffolding proteins and cytoskeletal proteins (van Zundert B. et al., Trends Neurosci. 27: 428-437, 2004, Elias G. M and R. A Nicoll, Trends Cell Biol. 17: 343-352, 2007).

PSD95 also has the ability to interact and co-localize with LRP1 (Niethammer M. et al., J. Neurosci. 16: 2157-2163, 1996; Martin A. M. et al., J. Biol. Chem. 283: 12004-12013, 2008). Interestingly, LRP1 protein expression level also increased significantly in the group treated with MDP. Accumulating evidences also suggest that LRP1 is a key player in AD pathology at the BBB level (Storck S. E. et al., J. Clin. Invest. 126: 123-136, 2016). Indeed, LRP1 is involved not only in Aβ endocytosis and cerebral degradation, but it is also a key player to eliminate Aβ across the BBB (Nazer B. et al., Neurobiol. Dis. 30: 94-102, 2008; Kanekiyo T. et al., J. Neurosci. 32: 16458-16465, 2012; Kanekiyo T. et al., J. Neurosci. 33: 19276-19283, 2013). Moreover, genetic risk factors for AD are linked to reduced clearance of Aβ via LRP1. More precisely, apolipoprotein E (apoE) E4 allele or the gene encoding the phosphatidylinositol-binding clathrin assembly (PICALM), has been reported to be a key factor in reducing clearance of Aβ via LRP1 (Bell R. D. et al., J. Cereb. Blood Flow Metab. 27: 909-918, 2007; Deane R. et al., J. Clin. Invest. 118: 4002-4013 2008; Zhao Z. et al., Nat. Neurosci. 18: 978-987, 2015). Additionally, LRP1 expression decreases in the brain and cerebrovascular system with age, indicating a potential target for treatment, as aging is the most prominent risk factor for AD (Kang D. E. et al., J. Clin. Invest. 106: 1159-1166, 2000; Silverberg G. D. et al., J. Neuropathol. Exp. Neurol. 69: 1034-1043, 2010). In parallel, the inventors observed a significant increase in COX2 expression levels in the MDP-treated group. While excessive COX2 activity plays a key role in neuroinflammation (Minghetti L., J. Neuropathol. Exp. Neurol. 63: 901-910, 2004), several studies have showed that it plays an important role in refinement of synaptic activity (Bosetti F. et al., J. Neurochem. 91: 1389-1397, 2004; Sang N. and C. Chen, Neuroscientist 12: 425-434, 2006). In this regard, involvement of COX2 in long-term synaptic plasticity and cognition has been supported from several behavioral tests (reviewed by Yang H. and C. Chen, Curr. Pharm. Des. 14: 1443-1451, 2008). The results obtained are in line with these studies since an improvement in memory deficits was observed while no significant increase in inflammatory markers in the brain was observed. Taken together, PSD95 and LRP1 are two key factors involved in MDP-derived memory improvement via enhancement of synapse function and vascular Aβ clearance. COX2 activity might also be a potential positive factor for cognition.

To further confirm the adhesion of monocytes to vascular Aβ-positive brain vessels, the inventors assessed MCP1 expression level since previous studies demonstrated MCP1-mediated monocyte recruitments (Simard, et al. 2006). MCP1 protein expression levels increased significantly in APP mice treated with MDP compared to controls. Interestingly, there was no significant change in nuclear factor kB (NF-kB). Since NF-kB is an inflammatory mediator involved in MCP production, it is believed that the increase in MCP1 expression level may not be dependent on the proinflammatory response. To further explore this phenomenon, the inventors next analyzed the endothelial inflammatory biomarkers, VCAM-1 and ICAM-1 (Chakraborty, et al. 2017). While VACM-1 showed no significant changes, a significant ICAM-1 decrease in APP mice treated with MDP compared to controls was observed. Consistent with the inventor's own observations, these results indicate that MDP treatments favor chemotactic gradients to allow recruitment of monocytes/macrophages to the brain vascular system without being associated with neuroinflammation.

Finally, the inventors investigated whether MDP-mediated shifting towards Ly6C^{low} monocytes could drive vascular Aβ clearance via Aβ uptake by these cells. Using live intravital two-photon microscopy in APP/PS1/CX3CR1 mice, it was observed that crawling GFP⁺ cells are significantly more frequent in blood vessels containing small Aβ aggregates in APP/PS1/CX3CR1^{gfp/+} mice treated with MDP than those treated with vehicle. It was also found that these crawling patrolling monocytes are selectively attracted to small Aβ aggregates present on APP/PS1/CX3CR1^{gfp/+} cortical blood vessels in response to MDP administrations. Collectively, these results provide direct in vivo evidence that MDP is a powerful drug to polarize Ly6C^{high} into Ly6C^{low} monocytes, which then patrol Aβ-containing small blood vessels for an efficient clearance of this toxic protein from the brain, explaining the delay of the onset of symptoms of AD and its improved treatment presented herein.

The findings reported herein demonstrate selective immunomodulatory effects of MDP on neurodegenerative diseases, such as MS and AD. Medications that solely target specific monocyte subsets and monocyte-derived macrophages with mild immunomodulatory effects in disease of the CNS without triggering microglial activation are rare. Here, the inventors have shown the therapeutic effects of MDP administration in EAE mouse model of MS, as well as in an AD mouse model. Furthermore, solid evidences are being provided herein, indicating the potential of MDP in terms of maintaining its therapeutic effect via regulating monocyte subsets in long term administration (both in WT and APP model). Taken together, these results suggest that MDP may be beneficial in both the early and progressive phase of MS, as well as early phase and to some extent late phases of AD.

In the present application, it is demonstrated in vivo in the mouse model for Alzheimer's disease that treatment with a NOD2 agonist increases the number of patrolling monocytes, which can then act as scavengers of Aβ. Further, as will be seen below, treatment with MDP increases expression of low density lipoprotein receptor-related protein 1 (LRP1), to increase the transport of amyloid beta (Aβ) from the abluminal to the luminal side of the blood brain barrier (BBB), where patrolling monocytes are awaiting to play their scavenger role. In the end, MDP treatment reduces amyloid beta and help improve or slow down cognitive impairment associated with AD. As such, the results provided herein demonstrate that agonists of NOD2 can be useful for treating AD, improving AD, or for delaying its onset.

### Mice

Animal experiments were performed according to the Canadian Council on Animal Care guidelines, as administered by the Animal Welfare Committee of Universite Laval. All efforts were made to reduce the number of animals used and to avoid their suffering. Three and six months old male APPswe/PS1 transgenic mice harboring the human presenilin I (A246E variant) and the chimeric mouse/human Aβ precursor protein (APP695swe) under the control of independent mouse prion protein (PrP) promoter elements [B6C3-Tg(APP695)3Dbo Tg(PSEN1)5Dbo/J] (Jackson ImmunoResearch Laboratories Inc.) were maintained in a C57BL/6J background. Mice were housed and acclimated to standard laboratory conditions (12-hour light/dark cycle/lights on at 7:00 AM and off at 7:00 PM) with free access to chow and water.

### Mouse Treatment

### MS Models

### Cuprizone Diet and MDP Treatment

Thirty 6 to 8-weeks-old C57BL/6J male mice were fed with either a normal chow (n=10) or CPZ-supplemented chow (n=20). 0.2% wt/wt CPZ [bis-cyclohexylidene hydrazide=cuprizone]; Sigma Aldrich) was mixed with regular ground chow and fed to experimental animals for 5 weeks. The food was changed every 2 days and food intake was monitored throughout the protocols. Control animals were fed with regular ground chow and manipulated as often as CPZ-fed mice. During the 5 weeks of diet, mice were injected three times per week with either MDP (N-acetylmuramyl-L-alanyl-D-isoglutamine) diluted in saline (10 mg/kg) or vehicle (saline 0.9%).

### EAE Induction and MDP Treatment

Fifty-seven 10-weeks-old male C57BL/6J mice as well as twelve 10-weeks-old male NOD2^{-/-} mice were used to study the impact of MDP treatment in the EAE model. EAE was induced by subcutaneous injection of mice with 2×100 μL of an emulsion containing CFA (complete Freud adjuvant), 1 mg Mycobacterium tuberculosis extract H37-Ra (Difco), and 100 μg MOG35-55 (MEVGWYRSPFSRVVHLYRNGK) along with an intraperitoneal injection of 200 ng pertussis toxin (PTX; List Biological Laboratories) on day 0 (immunization phase). On day 2, mice received a second intraperitoneal injection of PTX, followed 24 hour later by the first injection of MDP diluted in saline (10 mg/kg) or vehicle (saline 0.9%). MDP or vehicle were administered every 2 days. Animals were monitored daily for development of EAE according to the following criteria: 0, no disease; 1, decreased tail tone; 2, hind limb weakness or partial paralysis; 3, complete hind limb paralysis; 4, front and hind limb paralysis; 5, moribund state. To evaluate circulating immune cell subsets, blood samples were collected from the submandibular vein and kept in ethylenediaminetetraacetic acid (EDTA) coated vials (Microvette® K3E, Sarstedt, Montreal, QC, Canada) 7 and 21-days post-immunization. Mice were then sacrificed at 21-days post-immunization. To study the cerebral subsets of immune cells, mice were sacrificed 12-days post-immunization.

### APP Model and MDP Treatment

APP_{Swe}/PS1 expressing the chimeric mouse/human amyloid precursor protein (Mo/HuAPP695swe) and a mutant human presenilin 1 (PS1-dE9) under the control of independent mouse prion promoter elements [B6.CgTg(APPswe,PSEN1dE9)85Dboa]. A total of fifty-five 3-months old male APPswe/PS1 transgenic mice and twenty-five age matched C57BL/6J mice (WT) were utilized. Mice were injected two/three times per week with either MDP diluted in saline (10 or 20 mg/kg) or vehicle (saline 0.9%). By then, at 6 months, AD-related pathology has developed normally in the control mouse line.

### Triple-Transgenic Model

Mouse strains Cx3cr1gfp[B6.129P-Cx3cr1tm1Litt/J], expressing gfp under control of the chicken β-actin promoter and cytomegalovirus enhancer, and APPSwe/PS1 (see APP model section) were purchased from Jackson Laboratory (Bar Harbor, Me., USA). All mice were maintained in a pure C57BL/6J background, bred in house, and newborn pups were genotyped with PCR as advised by Jackson Laboratory protocols. Only males were used in the experiments. Mice injected four times for one week with either MDP diluted in saline (10 mg/kg) or vehicle (saline 0.9%).

### Flow Cytometry

Blood samples were collected from the submandibular vein and kept in EDTA coated vials on a rotator for <1 h. Flow cytometry analysis was performed as described by Lampron A. et al. (J. Exp. Med. 212: 481-495, 2015) and Lessard A. J. et al. (Cell Rep. 20: 1830-1843, 2017) for extracellular and by Brunet A. et al. (Eur. J. Immunol. 46: 2789-2800, 2016) for intracellular staining, respectively. FACS and data acquisition were performed using SORP LSR II™ and FACSDiva™ softwares (both from BD), respectively. Results were analyzed with the FlowJo™ software (v10.0.7).

Briefly, for extracellular staining, 50 μL of total blood was diluted with 35 μL of DPBS without Ca²⁺ or Mg²⁺ and incubated 15 min on ice with purified rat anti-mouse CD16/CD32 antibody (Mouse BD Fc Block; BD). Cells were then labeled at 4° C. during 40 min with the following rat anti-mouse antibodies: V500-conjugated anti-CD45 antibody (1/100, BD BioScience), AF700-conjugated anti-CD11b antibody (1/100, eBioscience), APC (allophycocyanin)-conjugated anti-CD115 antibody (1/100, eBioscience), V450-conjugated anti-Ly6C antibody (1/100, BD BioScience) and PE-conjugated anti-Ly6G antibody (1/100, eBioscience), FITC-conjugated anti-CD19 (1/100, eBioscience), PE-Cyanine5-conjugated anti-CD3 (1/100, eBioscience), PerCP-Cyanine5.5-conjugated anti-CD4 (1/100, eBioscience), PE-CF594-conjugated anti-CD8 (1/100, BD BioScience) and Live/Dead Fixable Blue Dead Cell Stain (Invitrogen, Paisley, UK). Next, red blood cells were lysed with 1.5 mL of 1× Pharm Lyse™ buffer (BD BioScience) during 20 min at room temperature, and the remaining leukocytes were washed and resuspended with DPBS without Ca²⁺ and Mg²⁺. More information about the procedure can be found at Thériault P. et al. (Oncotarget 7: 67808-67827, 2016).

For intracellular staining, briefly, 50 μL of total blood diluted with 600 μL of ACK lysis buffer and incubated 5 min at room temperature (RT). The blood was then centrifuged at 350×g for 5 min at RT. The supernatant was removed and the pellet was diluted in 3 mL cold PBS, then washed at 4° C., resuspended in 1 mL cell activation cocktail mix, and incubated for 4 h at 37° C. and 5% CO₂. In next step, cells were washed in 200 μL of dPBS or HBSS1× and spun at 1800 RPM for 3 min, then cells were resuspended in 100 μL of CD16/32 and incubated for 10 min on ice. Cells were then labeled at 4° C. during 40 min with the following rat anti-mouse antibodies: V500-conjugated anti-CD45 antibody (1/100, BD BioScience), FITC-conjugated anti-CD4 antibody (1/100, BD BioScience), PECF594-conjugated anti-CD8 antibody (1/100, BD BioScience), PerCPCy 5.5-conjugated anti-CD25 antibody (1/100, BD BioScience), PECY7-conjugated anti-CD3 antibody (1/100, BD BioScience), Live/Dead Fixable Blue Dead Cell Stain (Invitrogen, Paisley, UK). Next, the labeled cells were centrifuged and washed in 200 μL of dPBS or HBSS1×. Then, 200 μL Fixation/Permeabilization 1× was added to the cells which were then incubated for 20 min at RT. Next, the cells were washed and re-suspended in 100 dPBS and incubated overnight at 4° C. The next day, cells were centrifuged and 100 μL of permeabilization buffer 1× was added. Cells were then washed and the permeabilization buffer 1× was added again. In next step, cells were centrifuged, 100 μL of permeabilization buffer added, and cells were then labeled at 4° C. during 20 min with the following rat anti-mouse antibodies: eFluor 660-conjugated anti-Foxp3 antibody (1/100, BD BioScience) and PE-conjugated anti-IL-17 antibody (1/100, BD BioScience). Next, cells were centrifuged washed again with 100 μL of permeabilization buffer, and resuspended in 200 μL of 1× dPBS. More details about the procedure can be found in Brunet A. et al. (Eur. J. Immunol. 46: 2789-2800, 2016).

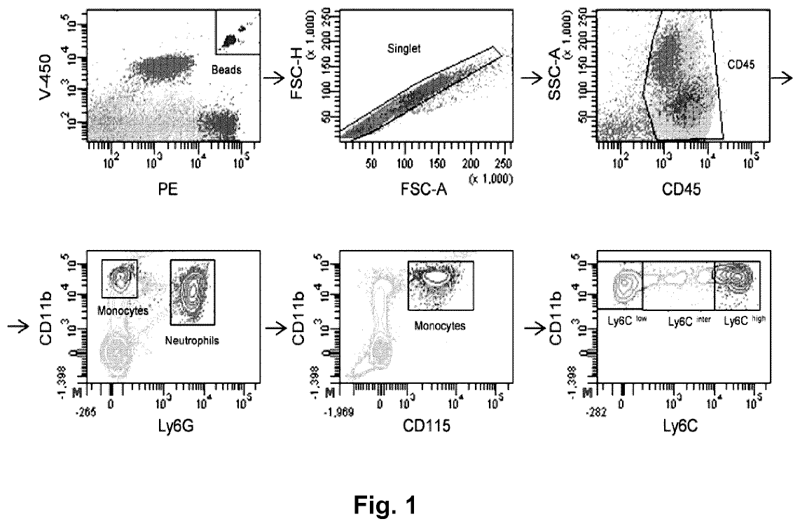

FIG. 1 represents the gating strategy for CD11b⁺ CD115⁺ monocyte and Ly6C monocyte subsets for all experiments and mouse models.

To identify absolute counting of cell populations, 123count eBeads™ were gated. Bead population excluded and doublet discrimination are performed with a singlet gate (FSC-H/F SC-A dot blot). Dead/live analysis was performed for CNS samples. Next, CD45⁺/CD11b⁺/Ly6G⁺ cells were considered as neutrophils. Neutrophil cell population was gated out. Next monocytes were identified with CD45, CD11b and CD115 expression. Monocyte subsets were further subdivided in three populations based on the expression of Ly6C: Ly6C^{high}, Ly6C^{int} and Ly6C^{low}, which correspond respectively to inflammatory, intermediate and patrolling monocytes.

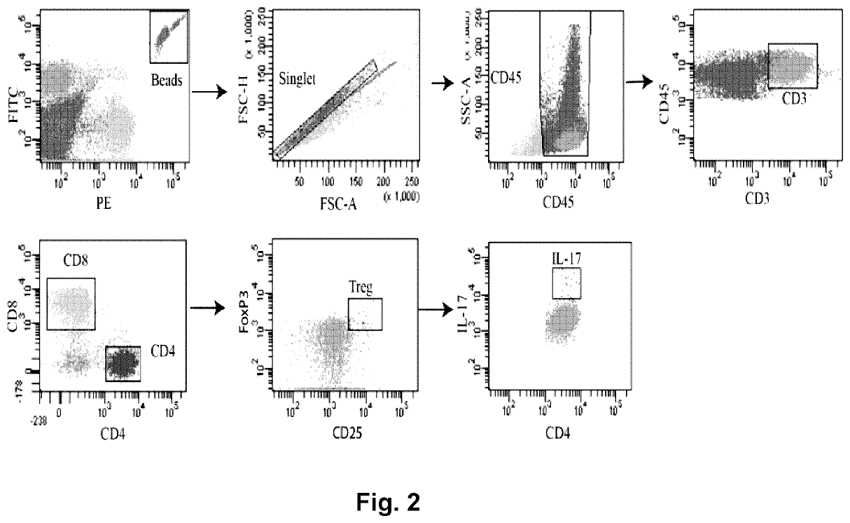

FIG. 2 represents the gating strategy for T cell subsets for all experiments and mouse models.

To identify absolute counting of cell population, 123count eBeads™ were gated. Bead population excluded and doublet discrimination are performed with a singlet gate (FSC-H/FSC-A dot blot). Dead/live analysis was performed for CNS samples. Next, CD45⁺/CD3⁺ cells were considered as CD3⁺. CD3⁺ were further subdivided in two populations based on the expression of CD4 and CD8. Next, Treg were identified with CD4⁺/Foxp3⁺/CD25 expressions. IL-17 was identified with CD4 and IL-17 expressions. IL-17⁺ CD8⁺ T cells were identified with the same strategy.

### CNS Flow Cytometry

EAE mice were deeply anesthetized via an i.p. injection of a mixture of ketamine hydrochloride and xylazine and then perfused intracardially with ice-cold dPBS. CNS were extracted and immediately homogenized for cell isolation. The same blood sample panels were used for extracellular and intracellular staining. FACS and data acquisition were performed using SORP LSR II and FACSDiva software (both from BD), respectively. Results were analyzed with the FlowJo software (v10.0.7).

Brain tissues were transferred to 3 mL Accutase (Sigma-Aldrich)+60 μL and DNase I 5 mg/mL (Sigma-Aldrich) and incubated for 20 min at 37° C. After homogenization, cells were passed through a 70 μm cell strainer and washed with HBSS. An additional 5 mL of HBSS 1× was added to the cells which were then centrifuged at 350×g, for 10 min at 4° C. Next, the pellets were resuspended in 8 mL of 30% Percoll, and centrifuged 20 min at 2500 RPM, at RT. Pellets were resuspended in 1 mL HBSS 1× and transferred to a new polypropylene tube through a cap filter tube 35 μm. 6 mL of dPBS was added to the cells which were then centrifuged at 350×g for 10 min at 4° C. Then, pellets were resuspended in 200 μL HBSS 1×. 100 μL was used for surface staining and 100 μL for intracellular staining. Surface and intracellular staining were performed as described above.

### Two-Photon Intravital Microscopy Imaging

### Mouse Strains

Mouse strains Cx3cr1gfp[B6.129P-Cx3cr1tm1Litt/J], expressing gfp under control of the chicken β-actin promoter and cytomegalovirus enhancer, and APPSwe/PS1 expressing the chimeric mouse/human amyloid precursor protein (Mo/HuAPP695swe), and a mutant human presenilin 1 (PS1-dE9) under the control of independent mouse prion promoter elements [B6.CgTg(APPswe,PSEN1dE9)85Dbo/J] transgenic mice were purchased from Jackson Laboratory (Bar Harbor, Me., USA). All mice were maintained in a pure C57BL/6J background, bred in house, and newborn pups were genotyped with PCR as advised by Jackson Laboratory protocols. Only males were used in the experiments. Animals were acclimated to standard laboratory conditions as previously described with ad libitum access to mouse chow and water. All animal procedures were conducted according to the Canadian Council on Animal Care guidelines, as administered by the Animal Welfare Committee of Universite Laval. In the Cx3cr1gfp/+ mouse, microglia, perivascular macrophages and monocytes, which all express CX3CR1, are GFP+.

### Cranial Window Preparation for Chronic Intravital Imaging

Craniotomy and cranial window preparation were performed as previously described with minor modifications (Mostany and Portera-Cailliau, 2008). Briefly, mice were anesthetized with isoflurane and the surgical site was shaved and sterilized with 2% chlorhexidine, 70% ethanol, and providone iodine. Animals were placed on a stereotaxic apparatus (Kopf Instruments, Tujunga, Calif., USA) and the ophthalmic ointment Lacri-Lube™ (Allergan, Markham, ON, CAN) was applied once the head was secured. Part of the scalp was removed, the right parietal bone was gently scraped with a scalpel blade and thinned by drilling a 6 mm wide circle so the bone flap could be delicately lifted up with small forceps. Occasional bleedings were stopped by applying Gel foam (Spongostan™, Johnson and Johnson) soaked with sterile saline. A 5 mm round glass coverslip (Electron Microscopy Sciences, Hatfield, Pa., USA) was laid on the dura mater and fixed with cyanoacrylate glue. The remaining surface of the skull was covered with dental acrylic and allowed to dry completely. Animals were maintained at 37° C. throughout the procedure and 1 ml of sterile saline was administered subcutaneously after the surgery. Animals were also given 0.15 mg of carprofen (Pfizer, Kirkland, QC, Canada) subcutaneously (s.c.) QD for 4-days post-surgery. Mice were allowed to recover during at least 3 weeks before intravital imaging experiments were initiated.

### Microinjection in the Cerebrospinal Fluid (CSF) Via the Cisterna Magna

Since a significant portion of subarachnoid CSF reaches perivascular spaces and gradually diffuses throughout the brain parenchyma (Iliff et al., 2012), Congo Red dye was injected into the cisterna magna to stain Aβ aggregates. The CSF was reached according to the procedure described by Liu and Duff (Liu and Duff, 2008). In short, mice were anesthetized with an intraperitoneal injection of a diluted mixture of ketamine (18.2 mg/kg) and xylazine (1.8 mg/kg) and placed on a stereotaxic instrument with the head inclined downward (≈140° from the body). Under a dissection microscope, a sagittal incision was performed below the occiput and the cisterna magna was exposed by gently spreading apart the muscles of the neck. Next, 4 μL of 0.1% Congo Red (Ricca Chemical Company, Tex., USA) was injected in the CSF via the cisterna magna at a rate of 1 μL/min through a 29G caliber needle connected to a 10 μL microsyringe (Hamilton, Reno, Nev., USA) mounted on an UltraMicroPump controlled by a Micro4 unit (World Precision Instrument, Sarasota, Fla., USA). Finally, the needle was removed incrementally over 2 min after the injection, the neck muscles were realigned, and the skin was sutured. Intravital imaging was performed as described by Michaud J. P. et al. (Proc. Natl. Acad. Sci. USA 110: 1941-1946).

### Sacrifices

Mice that received cuprizone-supplemented chow or normal chow, as well as EAE mice were deeply anesthetized with ketamine/xylazine and sacrificed via intracardiac perfusion with 0.9% saline followed by 4% paraformaldehyde (PFA) pH 7.4. The brains were then retrieved, post-fixed 10-24 hrs in 4% PFA pH 7.4, and transferred in 4% PFA pH 7.4+20% sucrose for a minimum of 15 hours. APP mice were perfused with 0.9% saline. Brains were retrieved and one hemisphere was snap-frozen for protein extraction while the other hemisphere was fixed in 4% PFA pH 7.4+20% sucrose. Brains were sliced in coronal sections of 25-μm thickness with a freezing microtome (Leica Microsystems), serially collected in anti-freeze solution and kept at −20° C. until usage.

### Post-Mortem Analysis

### Histochemical Immunostaining

Brain sections were washed four times for 5 min in KPBS and then blocked in kPBS containing 1% BSA, 4% NGS, and 0.4% Triton X-100™. The slices were then incubated overnight at 4° C. with the primary antibody anti-Olig2 (rabbit, 1:1000; Millipore) and anti-Iba-1 (rabbit, 1:1000; DAKO). After washing the sections four times for 5 min in KPBS, tissues were incubated in the appropriate secondary antibody (biotinylated goat anti-rabbit IgG; 1:1500, Vector Laboratories) for 2 h at RT. Following further washes in KPBS and 1 h-long incubation in avidinbiotin peroxidase complex (ABC; Vector Laboratories) to reveal the staining, the sections were then incubated in 3,30-diaminobenzidine tetrahydrochloride (DAB; Sigma). The sections were mounted onto Micro Slides Superfrost plus glass slides, dehydrated, and then coverslipped with DPX mounting media.

### Immunofluorescence

Brain sections were washed four times for 5 min in KPBS and then blocked in KPBS containing 1% BSA, 4% NGS, and 0.4% Triton X-100™. The tissues were incubated overnight at 4° C. with the primary Iba-1 antibody (1:2000; Wako Chemicals) and monoclonal anti-A13 (6E10, 1:3000; Covance). After washing four times for 5 min in KPBS, the tissue was incubated in the appropriate secondary antibody (IgG anti-mouse Alexa 488; Thermofisher and IgG anti-rabbit CY3; Jackson Immunoresearch) for 2 h at RT. Following further washes in KPBS and incubation with DAPI, the sections were mounted onto Micro Slides Superfrost Plus glass slides and coverslipped with Fluoromount-G (Electron Microscopy Sciences).

### Image Acquisition and Analyses

Image acquisition of fluorescence-stained images was performed using a Zeiss LSM800™ confocal microscope supported by the Zen™ software (2.3 system) using the 4× and 40× lenses as described previously (Laflamme N. et al., Front. Cell. Neurosci. 12: 178, 2018). Number of 6E10, Iba-1 associated to plaques were quantified by unbiased stereological analysis (Thériault P. et al., Oncotarget 7: 67808-67827, 2016) using Stereo Investigator software (version 6.02.1, MicroBrightfield) attached to a Nikon C80i™ microscope equipped with a motorized stage (Ludl) attached to Microfire CCD color camera (Optronics). For each animal, 4-6 sections were analyzed.

### Black Gold Staining

Brain sections were washed three times for 10 min in cold KPBS and mounted onto Superfrost slides glass slides. The slides were pre-warmed 30 min at 65° C. on a slide warmer, washed once with warm KPBS, followed by an incubation in 0.3% Black Gold (EMD Millipore) diluted into 0.9% NaCl for 30 minutes. After this time, slides were washed in warm KPBS, then in warm sodium thiosulfate for 3 min, and then transferred into KPBS. All steps were performed at 65° C. Finally, slides were dehydrated in alcohol (ethanol 95%), cleared in xylene, and coverslipped with DPX. Using a QImaging camera, 8-bit grayscale TIFF images of the regions of interest were taken in a single sitting for Cuprizone model, with the same gain/exposure settings for every image. To quantify the level of demyelination/myelination, these images were imported into ImageJ and myelination of a given area was measured as the surface proportion of staining intensity above a determined threshold.

### In Situ Hybridization

In situ hybridization was performed as described previously (Laflamme N. and S. Rivest, FASEB J. 15: 155-163, 2001) on all sections of the brain, starting from the end of the olfactory bulb to the end of the cortex. ³⁵S-labeled complementary RNA probes for Trem2, Tlr2, and Pdgrfa were used for in situ hybridization. Films were then scanned using an Epson Perfection v850 Pro™ scanner supported by the SilverFast™ software (version 8.8.Or6). Area and intensity of positive hybridization signals were densitometrically measured on all brain sections using ImageJ software (Version 2.0.0-rc-43/1.51n). Each value was corrected for background signal by subtracting the OD value measured at a brain area devoid of positive signal (for a detailed protocol, see Laflamme N. et al., J. Neurosci. 19: 10923-10930, 1999).

Soluble Aβ₁₋₄₂/Aβ₁₋₄₀ELISA

Brain levels of soluble Aβ₁₋₄₂ and Aβ₁₋₄₀ were quantified by using the Human Amyloid β42 and Human Amyloid β40 Brain ELISA kits (Millipore, Billerica, Mass., USA). Experimental procedure was performed according to the manufacturer's instructions (Michaud J.-P. et al., Proc. Natl. Acad. Sci. USA 110: 1941-1946, 2013).

### Western Blot Analysis

Hippocampus and cortex brain protein were lysates as previously described (Michaud J.-P. et al., Proc. Natl. Acad. Sci. USA 110: 1941-1946, 2013). Proteins were then loaded in 4-15% agarose precast gels (Bio-Rad) and electroblotted onto 0.45 μm Immobilon PVDF membranes. Membranes were immunoblotted with various primary antibodies as described in Table 1, followed by the appropriate horseradish peroxidase (HRP)-conjugated secondary antibodies and revealed by enhanced chemiluminescence plus (ECL™) solution (GE Healthcare Life Sciences). Quantification was done by determining integrative density of the bands using Thermo Scientific Pierce myImage™ Analysis Software v2.0. Optical values were normalized over actin. Listed in Table 1 are the antibodies used for immunoblot analyses and all related information including name of company, molecular weight, species, secondary antibodies and dilution rates.

TABLE 1
Antibodies used for immunoblot analyses

| | | | | | | Secondary | |
|---|---|---|---|---|---|---|---|
| | | Molecular | | | | antibody | |
| Antibody | Supplier | weight | | Species | Dilution | dilution | Notes |
| Actin | Millipore | 42 | kDa | Mouse | 1/50 000 | 1/20 000 | |
| APP | Millipore | ≈100 | kDa | Mouse | 1/2000 | 1/5 000 | Antigen retrieval |
| | | | | | | | (Michaud et al., 2013) |
| Cox-2 | Santa Cruz | ≈75 | kDa | Goat | 1/1 000 | 1/5 000 | |
| Iba-1 | Wako | 18 | kDa | Rabbit | 1/1 000 | 1/5 000 | |
| ICAM | Santa Cruz | 85-110 | kDa | Goat | 1/500 | 1/1 000 | |
| LRP1 | CEDARLANE | 85 | kDa | Rabbit | 1/1 000 | 1/40 000 | |
| MCP1 | Cell Signaling | 13 | kDa | Rabbit | 1/1 000 | 1/5 000 | |
| PSD95 | Neuromab | 95 | kDa | Mouse | 1/2 000 | 1/20 000 | |
| Synaptophysin | Thermo Fisher | 34 | kDa | Mouse | 1/10 000 | 1/100 000 | |
| | Scientific | | | | | | |
| Trem2 | R&D Systems | 40 | kDa | Rabbit | 1/500 | 1/2 000 | Antigen retrieval |
| | | | | | | | (Michaud et al., 2013) |
| VCAM | Santa Cruz | 90-100 | kDa | Rabbit | 1/1 000 | 1/10 000 | |
| NFkB p50 | CEDARLANE | 50 | kDa | Rabbit | 1/1 000 | 1/10 000 | |

### Behavioral Tests

### Open Field

Open field performed to evaluate anxiety-like behaviors, exploration habits and also locomotor activity is as described by Hui et al. (Brain Behay. Immun. 73: 450-469). Each mouse was individually recorded and analyzed by ANY-maze system.

### Novel and Spatial Object Recognition

Novel object recognition (NOR) task, and also spatial object recognition (SOR) were performed with the open field platform according to Hui et al. (Brain Behay. Immun. 73: 450-469). Each mouse was individually recorded and analyzed by ANY-maze system.

T-Water Maze

The T-water maze assay was performed according to Guariglia et al. (J. Neurosci. Meth. 220: 24-29). The pool was filled with 23° C. (±1° C.) water to a depth of 13 cm, which was 1 cm above the surface of the platform. Mice were trained to swim to a particular arm of the T and to remain on a submerged platform for 5 s. Mice had to complete six out of eight trials without error for two consecutive days out of three days to reach the learning criterion. The same criterion was considered for reversal phase.

### Two-Photon Intravital Microscopy Imaging

Prior to the imaging session (5-15 min), blood vessels were labeled by Qdot 705 (Qtracker705, 5% w/v in PBS, Invitrogen, ON, Canada) administered via the tail vein. Animals were anesthetized with the same ketamine/xylazine mixture described above and were placed prone on a small stereotaxic instrument where they were maintained at 37° C. by a temperature controlling device (RWD, Life Science Co., ShenZhen, China). The cranial glass window was covered with few drops of water and intravital imaging was carried out with an Olympus FV1000 MPE™ two-photon microscope (Richmond Hill, ON, Canada) equipped with a Mai Tai DeepSee™ laser (Spectra-Physics, Newport Corp., Santa Clara, Calif., USA) tuned at 925 nm. All images were acquired using an Olympus Ultra 25×MPE™ water immersion objective (1.05 NA), with filter set bandwidths optimized for YFP (520-560 nm), Texas Red/DsRed (575-630 nm), and Qdot 705/800 (662-800 nm) imaging. PMT sensitivity and gain were set in order to obtain a maximal dynamic range of detection. Images were acquired at a zoom factor ranging from 1.0 to 1.5×, with the Olympus Fluoview™ software (version 3.0a). Kalman filtering was deactivated for time-lapse imaging and blood vessels were used as landmarks for chronic intravital imaging. All image processing was carried out with ImageJ (US National Institute of Health, Bethesda, Md., USA). The number of GFP-positive crawling cells into blood vessels was manually quantified over time. For details on two-Photon intravital microscopy imaging experiments, further details are provided in Michaud et al. (Cell Rep. 5: 646-653, 2013).

### Statistics

Data are expressed as the mean±SEM. Comparison between two groups were conducted using post hoc unpaired t tests, Wilcoxon rank-sum tests, or Wilcoxon-Mann-Whitney test. For EAE mice, regression analysis was also performed. Comparisons between more than two treatment groups were conducted using either one-way analysis of variance (ANOVA) or two-way repeated measures ANOVA, followed by Tukey's post-hoc test. Values were statistically significant if P<0.05. All analyses were performed using GraphPad Prism Version 6 for Windows (GraphPad Software, San Diego, Calif., USA) and SAS 9.4 (SAS Institute Inc., Cary, N.C., USA). All panels were assembled using Adobe Photoshop™ CS5 (version 12.0.4) and Adobe Illustrator™ CS5 (version 15.0.2).

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

### Example I

### MDP Administration in MS Models—Cuprizone-Induced Demyelinating Model

Systemic MDP Administrations Shifting Circulating Monocyte Population Towards Ly6C^{low} Monocyte Subsets in Mice Fed with Cuprizone-Supplemented Diet

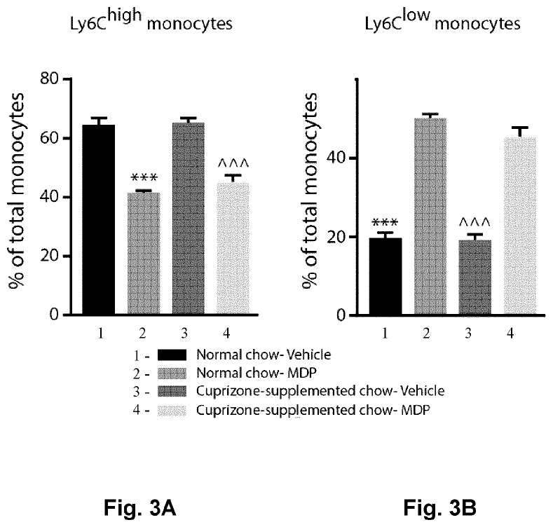

Microglia and monocyte-derived macrophages coordinate remyelination process via phagocytosis and inflammatory responses (Döring A. et al., J. Neurosci. 35: 1136-1148, 2015; Lampron A. et al., J. Exp. Med. 212: 481-495 2015). In this regard, previous study from our group showed phagocytic feature of Ly6C^{low} monocytes in CNS (Michaud J.-P. et al., Cell Rep. 5: 646-653, 2013). Immunomodulatory effects of MDP was first examined in the cuprizone (CPZ) model. Wild type mice were fed with normal chow or CPZ-supplemented chow during 5 weeks, and the peak of demyelination is observed between 4 and 5 weeks of diet. During the five weeks of CPZ intoxication, mice received MDP (10 mg/kg) or saline injections twice a week. Mice were followed-up throughout the experimental course to evaluate food intake as well as body weight. No differences were observed in food intake in any group. However, both groups fed with CPZ-supplemented chow exhibited weight loss. At the end of the CPZ intoxication, blood was collected and monocyte populations were examined. Compared to control groups, MDP treatments showed a significant increase in percentage of Ly6C^{low} monocytes and also significantly decreased in percentage of Ly6C^{high} monocytes in both groups of mice fed with CPZ-supplemented chow or normal chow. In particular, following MDP administrations, initial percentage of Ly6C^{high} monocytes which was about 60% in both normal food and CPZ groups decreased to 40%. In parallel, the percentage of Ly6C^{low} monocytes (20%) increased and reached to approximately 50% in both groups (FIGS. 3A and 3B).

Before ending the protocol, several behavioral tests were performed to explore if demyelination level induced by CPZ intoxication was associated with neurological alterations. It was observed that demyelination level was not reflected in different behavioral tests including ledge test, nesting behavior test, open field test, pole test and neurological exam in MDP or vehicle CPZ-supplemented chow groups.

Systemic MDP Administration does not Affect the Remyelination and Brain Inflammation

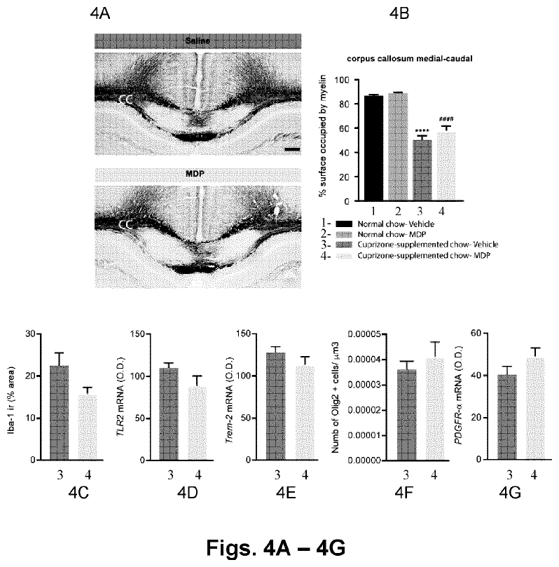

Next, it was investigated if the MDP-mediated immune regulation could affect the demyelination at histological level. CPZ intoxication leads to myelin loss in brain white matter. The corpus callosum, being the largest white matter region of the brain, is particularly susceptible to CPZ. Black Gold II staining (FIG. 4A) was performed in the brain tissue from mice that were fed with CPZ-supplemented chow and were treated with MDP or saline. Because more severe myelin depletion is expected in the medial-caudal area of the corpus callosum (Laflamme N. et al., Front. Cell. Neurosci. 12: 178, 2018), the inventors analyzed this region measuring the area occupied by myelin and did not observed any differences in the myelin levels following MDP treatments (FIG. 4B). Concomitantly to the myelin loss, a robust microglial response is observed in the corpus callosum of mice that were intoxicated with CPZ. Following the observation of shift in monocyte subsets in the periphery, it was then verified whether MDP is capable of modulating microglia, and their activation as well as their phagocytic properties. Following MDP treatment, mice that were exposed to CPZ did not show any modulation of the microglial response (FIG. 4C), its activation measured by TLR2 expression (FIG. 4D) or its phagocytic activity measured by TREM2 level (FIG. 4E). Finally, it was evaluated whether MDP could affect the number of oligodendrocytes and oligodendrocyte progenitor cells, measured by the expression levels of PDGFRα and even in this case, no difference was observed between the groups treated with MDP or saline (FIGS. 4F and 4G).

In summary, these results indicate that MDP administrations convert Ly6C^{high} to Ly6C^{low} monocytes in the CPZ model. However, it does not impact either the brain myelination level or the cerebral immune response. These results suggest that the peripheral immune response does not drive the remyelination process in mice exposed to CPZ.

### Example II

### MDP Administration in MS Model; EAE Model

### MDP-Treated Mice are Highly Resistant to the Onset of EAE Via Shifting Monocyte Subsets Towards Ly6C^{low} Monocytes and Regulating the Population of T Cell Subsets

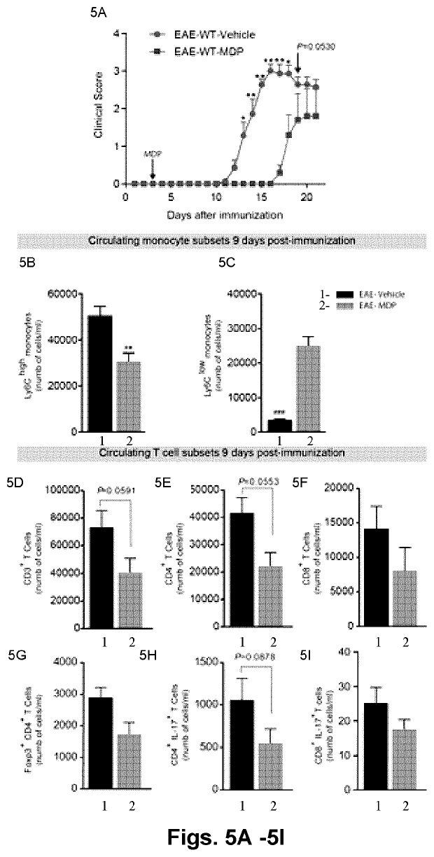

To address the potential therapeutic effect of MDP in the EAE model, the inventors assessed the EAE onset, disease progression and severity in mice treated with MDP (EAE-MDP) or vehicle (EAE-vehicle). Mice were immunized by subcutaneous injection of a MOG peptide emulsified in complete Freund's adjuvant and accompanied by pertussis toxin, as previously described herein. Animals were injected with MDP or saline two-days post-immunization. EAE-vehicle mice developed disease as characterized by ascending paralysis (Rangachari M. and V. K. Kuchroo, J. Autoimmun. 45: 31-39, 2013). Interestingly, EAE mice treated with MDP were protected from progression of diseases as measured by clinical scores and showed a delay in the day of onset (P≤0.0001) (FIG. 5A). The incidence of disease after EAE induction was lower in EAE-MDP than EAE-Vehicle. In addition, the number of mice that developed hind-limb paralysis after EAE immunization was reduced in the EAE-MDP group (Table 2).

TABLE 2
EAE progression in WT mice treated with vehicle or MDP

| | | Disease | Complete | | |
|---|---|---|---|---|---|
| | Disease | onset | hind-limb | Maximum | Total |
| Groups | incidence | (days) | paralysis | score | days |
| EAE/WT- | 5/5 (100%) | 11 | 100% | 3.5 | 21 |
| Vehicle | | | | | |
| EAE/WT- | 3/5 (60%) | 17 | 60% | 3 | 21 |
| MDP | | | | | |

To explore the mechanisms underlying the protecting properties of MDP in the EAE model, blood leukocyte subpopulations were quantified before disease onset (one-week post MDP injections or 9-days post immunization) by flow cytometry (FACS) analysis. It was observed that MDP administrations significantly increased Ly6C^{low} monocytes while decreasing Ly6C^{high} monocyte levels (FIGS. 5B and 5C). Interestingly, there is a tendency for a decreased number of T cell subsets comprising CD3⁺ T cells (P=0.0591) and CD4⁺ T cells (P=0.0553). Although not significant, number of CD8⁺ T cells numbers also showed a shift toward reduction (FIGS. 5D, 5E, and 5F). Finally, the inventors were interested to determine whether other T cell subsets were regulated upon MDP treatments. A slight reduction (not significant) was found in the number of CD4⁺ CD25⁺FoxP3⁺ regulatory T cells (Treg cells) in EAE-MDP mice. Interestingly, mice administrated with MDP showed a tendency (P=0.0879) toward decreasing in number of IL-17⁺ CD4⁺ T cells (FIGS. 5G and 5H). In addition to CD4⁺ T cells, there is evidence that IL-17⁺ CD8⁺ T cells contribute to pathology in EAE and are present in the cerebrospinal fluid (CSF) of patients with MS (Annibali V. et al., Brain 134: 542-554 2011; Huber M. et al., J. Clin. Invest. 123: 247-260, 2012). IL-17⁺ CD8⁺ T cells were reduced slightly (not significant) in MDP-treated group versus vehicle group (FIG. 5I). These results indicate a critical role of MDP in modulating monocyte subsets and to some extent T cell subsets in the EAE model.

Both groups of mice were then compared at 21-days post immunization when the EAE-Vehicle group stabilized as demonstrated by clinical scores while the EAE-MDP group just entered into the acute phase (Table 2). EAE mice that received MDP for 21 days exhibited a reduced number of Ly6C^{high} cells together with an increased number of Ly6C^{low} monocytes. In addition, the chronic treatment also slightly (not significant) reduced the number of T cell subsets, in particular CD3⁺, CD4⁺, and CD8⁺ T cells. Altogether, these results suggest that MDP administrations after onset of EAE keep shifting monocyte subsets towards Ly6C^{low} monocytes and slightly regulate T cell subsets during acute phase of EAE.

### Example III

### MDP Administrations Modulate Monocyte Subsets and Infiltrating of Ly6C^{high}, Ly6C^{low} Monocytes, T Cell Subsets, Ly6G⁺ Cells and CD19⁺ Cells in the CNS Before Onset of EAE

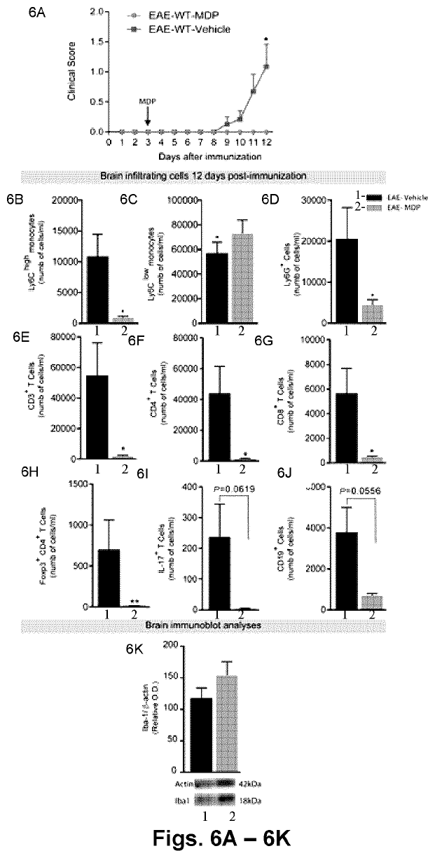

Next, it was then investigated to determine if the immunomodulatory effects of MDP on monocyte subsets, clinical scores and onset of EAE are correlated with regulation of infiltrating cells into the CNS. Mice were immunized and injected with MDP every 2 days as previously described herein. At 12-days post immunization, all EAE-Vehicle mice had developed EAE, whereas the EAE-MDP group showed no clinical symptom (P≤0.0001) (FIG. 6A and Table 3).

TABLE 3
EAE progression in WT mice treated with vehicle or MDP

| | | Disease | Complete | | |
|---|---|---|---|---|---|
| | Disease | onset | hind-limb | Maximum | Total |
| Groups | incidence | (days) | paralysis | score | days |
| EAE/WT- | 6/13 (46.1%) | 10 | 38% | 3.5 | 12 |
| Vehicle | | | | | |
| EAE/WT- | 0/13 (0%) | — | 0 | 0 | 12 |
| MDP | | | | | |

At this time point, FACS analysis of the CNS confirmed the drastic reduction in the number of Ly6C^{high} together with the increase in Ly6C^{low} monocytes (FIGS. 6B and 6C) as well as Ly6G⁺ cells (FIG. 6D).

To determine whether these findings were also reflected in disease-specific T cells, T cell subsets were analyzed. CD3⁺, CD4⁺, and CD8⁺ T cell numbers were significantly reduced in EAE-MDP compared to EAE-Vehicle group (FIGS. 6E, 6F, and 6G). In addition, the numbers of Foxp3⁺ regulatory T cells, IL-17⁺ CD4⁺ T cells and CD19⁺ cells were significantly reduced in MDP-treated group compared to the control (FIGS. 6H, 6I, and 6J). Interestingly, IL-17⁺ CD8⁺ T cells were not detected in the CNS of treatment and control groups.

Finally, it was then evaluated whether MDP modulated microglial response by measuring Iba-1 protein levels. Iba1 protein levels in the CNS showed no significant difference between the treatment and the control groups confirming that MDP regulates specifically systemic myeloid cell infiltration in EAE (FIG. 6K). These results together with the clinical scores indicates that MDP significantly delayed onset of EAE via the regulation of infiltrating monocyte and T cell subsets with no evidence of altering microglia.

### Example IV

### NOD2 Receptor Plays a Critical Role in MDP-Dependent Immune Modulation and EAE Resistance

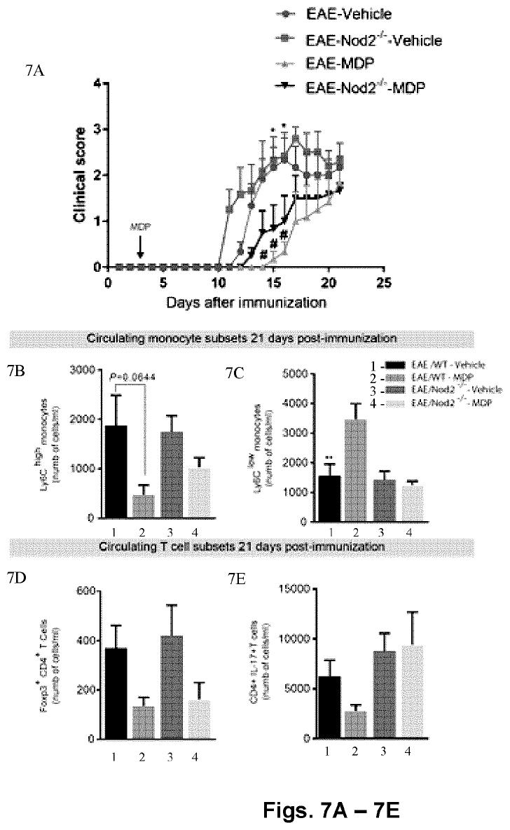

To address the role of NOD2 receptor in MDP-mediated EAE resistance, EAE was induced in both WT and NOD2^{-/-} mice and these mice were then injected with either saline or MDP every two days. The incidence of disease in EAE-NOD2^{-/-}-MDP was higher (100%) compared to the WT counterpart (66%) (Table 4). Moreover, the onset of disease was slightly earlier in EAE-NOD2^{-/-}-MDP compared to WT mice (FIG. 7A and Table 4). The severity of disease progression in EAE-NOD2^{-/-}-MDP seems slightly higher than the control group (EAE-MDP) (FIG. 7A). More importantly, the percentage of mice that developed hind-limb paralysis was higher in EAE-NOD2^{-/-}-MDP (83%) than control WT (50%) (Table 4).

TABLE 4
EAE progression in WT and NOD2−/−
mice treated with vehicle or MDP

| | | | Complete | | |
|---|---|---|---|---|---|
| | Disease | Days of | hind-limb | Maximum | Total |
| Groups | incidence | onset | paralysis | score | days |
| EAE/WT- | 6/6 (100%) | 12 | 100% | 3.5 | 21 |
| Vehicle | | | | | |
| EAE/WT- | 4/6 (66.6%) | 15 | 50% | 3.5 | 21 |
| MDP | | | | | |
| EAE/Nod2^{-/-}- | 5/5 (100%) | 11 | 83% | 3.5 | 21 |
| Vehicle | | | | | |
| EAE/Nod2^{-/-}- | 6/6 (100%) | 13 | 83% | 2 | 21 |
| MDP | | | | | |

Since NOD2^{-/-} mice did not respond as well as the WT group to MDP, it was then verified if peripheral cells were modulated by MDP 21-days post-immunization. MDP administrations modulated the number of Ly6C^{high} and LyC6^{low} monocytes in WT mice. As expected, no differences were observed in number of Ly6C^{high} and LyC6^{low} monocytes in NOD2^{-/-} mice treated with MDP (FIGS. 7B and 7C). In addition, the number of CD3⁺, CD4⁺, and CD8⁺ T cells did not change significantly in NOD2^{-/-} mice treated with MDP. No differences were also observed for Foxp3⁺ CD4⁺ T cells and IL-17⁺CD4⁺ T cells (FIGS. 7D and 7E). Altogether, these results suggest a critical role of NOD2 receptor in MDP-mediated immune resistance and innate immune modulation in the EAE model.

### Example V

### Ly6C^{low} Patrolling Monocytes are Increased in the Blood of APP Mice Following MDP Treatment

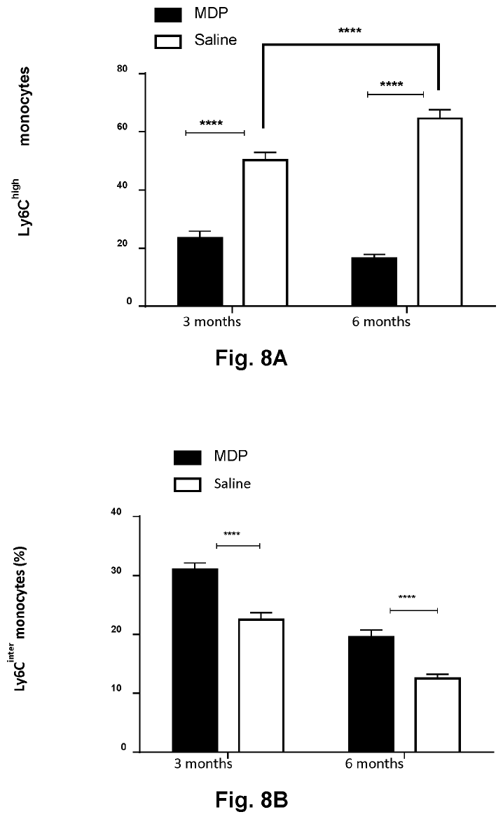

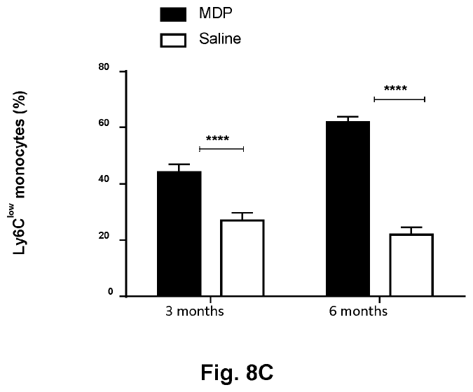

Three and six months old APP/PS1 mice were i.p. treated with MDP or saline every 72 hours for the period of 3 months. Blood samples were taken and the percentage of monocyte subsets was analyzed by flow cytometry, as described above. MDP injections decreased the number of Ly6C^{high} monocytes (FIG. 8A) while they increased the percentage of Ly6C^{low} monocytes (FIG. 8B) at both 3 and 6 months of age. As previously reported in wild-type mice, this mouse model of AD responded to the molecule in a very similar manner that is the switch of inflammatory monocytes into the patrolling subset of cells. Inflammatory monocytes are the direct target of MDP to convert them into patrolling monocytes via their intermediate phase (FIG. 8B) since they are the precursor cells. These data provide direct evidence that MDP has the ability to trigger the switch of inflammatory monocytes into the patrolling subset in this mouse model of AD.

### Example VI

### Chronic MDP Administration in a Mouse Model of AD Improves Cognitive Deficits

### High Frequency

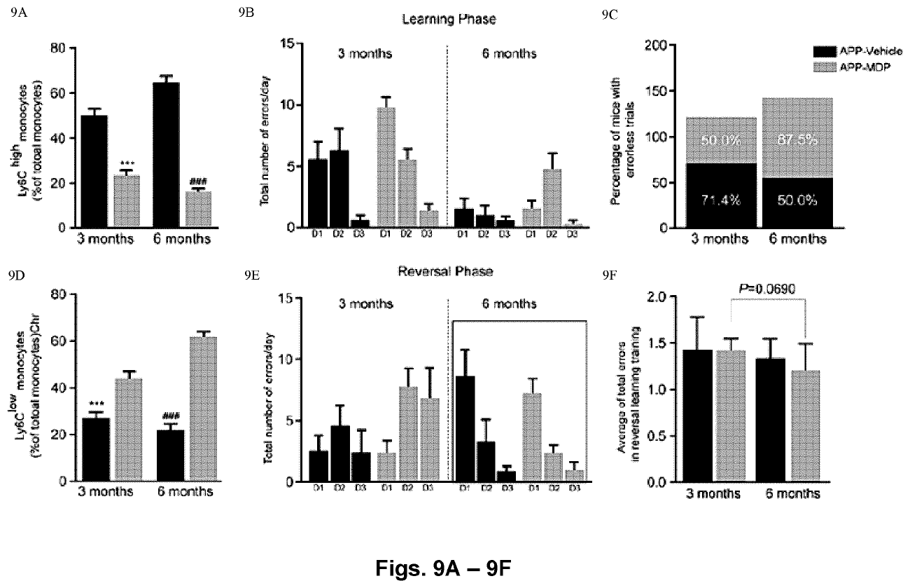

Ly6C^{low} monocytes are able to associate within Aβ-positive veins, but not arteries, internalize Aβ, and efficiently eliminate and transport Aβ microaggregates from the brain microvasculature to the blood circulation. Immunoregulatory of MDP in shifting monocyte subsets towards Ly6C^{low} prompted the inventors to assess potential therapeutic effects of MDP in APP mice. 3 month-old APP mice were chronically administered MDP twice a week (high frequency) in over 6 months period as previously described herein. The inventors then evaluated circulating monocyte subsets at both 3 and 6 months following the beginning of the injections. APP mice develop an Alzheimer-like phenotype at 6 months of age. In parallel, 4-6 months old APP mice develop accumulation of small and punctate A13 aggregates on specific blood vessels. Thus, these time points were chosen to evaluate whether MDP is capable of delaying disease onset (3 months following the first MDP injection) and maintain the phenotype over time (6 months after the first MDP injection). As with both MS-like models, the drug can modulate monocyte phenotype towards the Ly6C^{low} subsets, both at 3 and 6 months post-injections (FIGS. 9A and 9B). To assess whether MDP affects cognitive behavior, a water T-maze test was performed. It was observed that APP mice that received MDP did not significantly differ from their counterparts that received saline both during the learning and reversal phases of water T-maze test (FIGS. 9C and 9D). Nevertheless, a higher percentage of APP mice that received MDP did not make any error during the reversal phase 6 months after the first MDP injection (FIG. 9E). Additionally, these mice showed a tendency (P=0.0690) to have a lower number of total errors at the later time point (FIG. 9F). It is important to mention that no significant changes were observed in open field test results, indicating MDP treatments did not cause neither anxiety-like behaviors nor locomotor activity problem. Overall, these results show that monocytes are modulated in APP mice and the treatment improved the cognitive deficits of the mice when the disease is established.

### Low Frequency

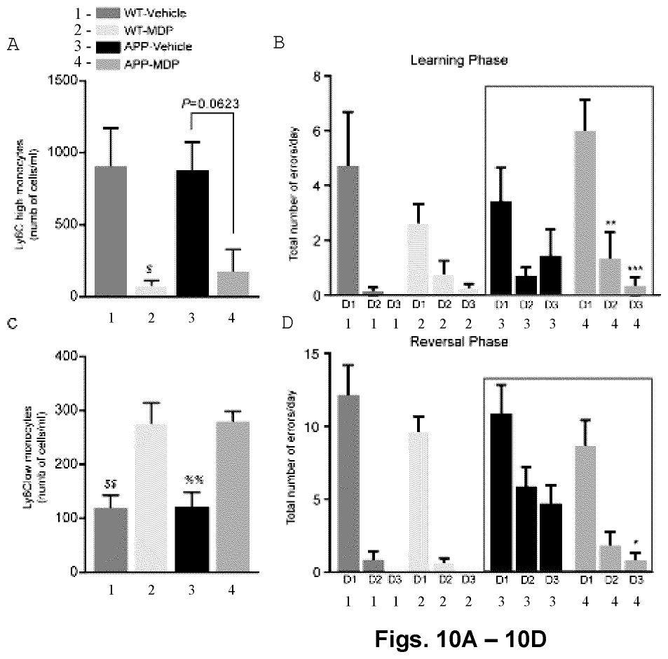

The inventors then tested whether chronic MDP injection in APP and WT mice at a lower frequency (once a week) over a 3-month period, as previously described herein. Similar to the high frequency MDP administration, a low frequency MDP administration shifting monocyte subsets towards the Ly6C^{low} phenotype (FIGS. 10A and 10B). Most importantly, it was observed that 3 months after the first MDP injection, the cognitive phenotype is improved both during the learning and reversal phase of the T-water maze (FIGS. 10C and 10D). In addition, the same results were observed as the high frequency protocol from the open field test. These results suggest that chronic administration of MDP at lower frequency is sufficient to delay the appearance of an Alzheimer or Alzheimer-like phenotype.

### Example VII

### MDP-Derived Memory Improvement is not Dependent on Change in Aβ Levels and Microglial Activation

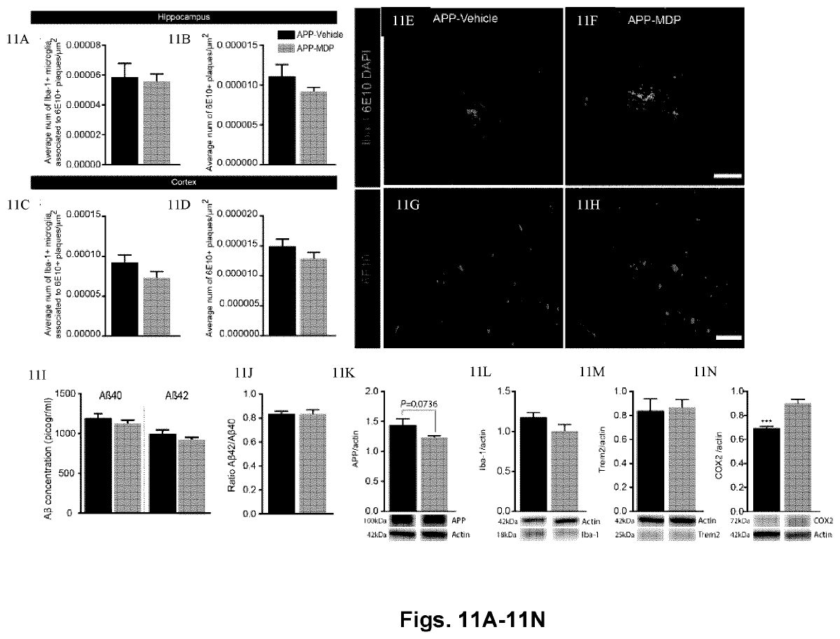

Microglial cells play a key role in AD pathogenesis by regulating Aβ levels in the brain via uptake and degradation processes. Therefore, MDP treatments were then evaluated for their impact on Aβ accumulation and microglia functions by measuring the number of Iba1-positive microglia associated to 6E10-positive plaques as well as the number of immunostained plaques in both the hippocampus and cortex of APP mice that received MDP or saline. No differences were observed between the two groups (FIGS. 11A to 11H).

Soluble Aβ40 and Aβ42 levels were then measured in cortex and hippocampus by specific ELISA immunoassays, and even in this case the results showed no significant difference between treatment and control groups (FIGS. 11I and 11J). As Aβ is produced through sequential cleavage of APP, catalyzed by β- and γ-secretase, the expression level APP was then measured by immunoblot and a tendency to decrease in the MDP-treated group (P=0.0736) (FIG. 11K) was observed. Finally, the expression levels of Iba1, TREM2, and NFkB were measured in the hippocampus of APP mice treated with MDP or saline and no differences were observed (FIGS. 11L and 11M). Finally, despite that no modulation of these inflammatory markers could be observed, an increase in COX2 levels in MDP-treated mice was noted (FIG. 11N). Altogether, these results indicate that the memory/learning improvements observed in behavioral tests are not dependent on the Aβ burden or microglial activation, suggesting other factor(s) involved in MDP-mediated cognitive improvement.

### Example VIII

### MDP-Derived Memory Improvement May be Mediated by Modification of Synaptic Function and Aβ Vascular Clearance

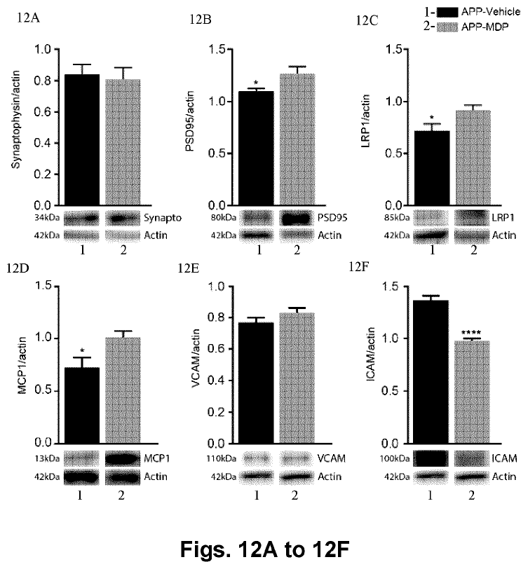

The inventors then investigated if MDP-derived memory improvement is dependent on improvement in synapse formation. Hence, pre- and postsynaptic puncta (synaptophysin and PSD95) in treatment and control groups were quantified. While immunoblot analysis of synaptophysin showed no significant difference (FIG. 12A), it was noted that PSD95 levels were significantly increased in APP-MDP mice compared to those of control (FIG. 12B). The low density lipoprotein receptor-related protein-1 (LRP1) level was also analyzed as this protein interacts and co-localizes with PSD95 for synapse formation and is a key player to eliminate Aβ across the BBB. Interestingly, LRP1 protein expression levels also increased significantly in the group treated with MDP (FIG. 12C). Altogether, these results indicate that memory improvement mediated by MDP may depend on enhancement of synaptic plasticity and vascular Aβ clearance.

### Effect of MDP on Key Proteins Involved in Cerebrovascular Monocyte Adhesion

Because there were no differences at the microglial level, it was then evaluated whether proteins normally associated with monocyte recruitment and vascular adhesion were modulated following MDP treatments. Monocyte chemoattractant protein-1 (MCP1) has a key role in the recruitment of monocytes along the cerebrovascular elements and is significantly increased in the brain of mice treated with MDP (FIG. 12D). Interestingly, no significant change was found for nuclear factor kB (NF-kB, P50). Then, the expression levels of vascular cell adhesion molecule-1 (VCAM-1) and intercellular adhesion molecule-1 (ICAM-1) were evaluated. VCAM-1 showed no significant changes, whereas a reduction in the expression level of ICAM-1 was observed in mice treated with MDP when compared to the control group (FIGS. 12E and 12F). Consistent with previous observations, these results indicate that MDP has no clear modulatory neuroinflammatory effects in the brain, but can modulate the expression levels of chemotactic factors.

### Example IX

### MDP-Derived Shifting Monocytes Towards Ly6Clow Monocytes are Selectively Attracted to Small Aβ Aggregates

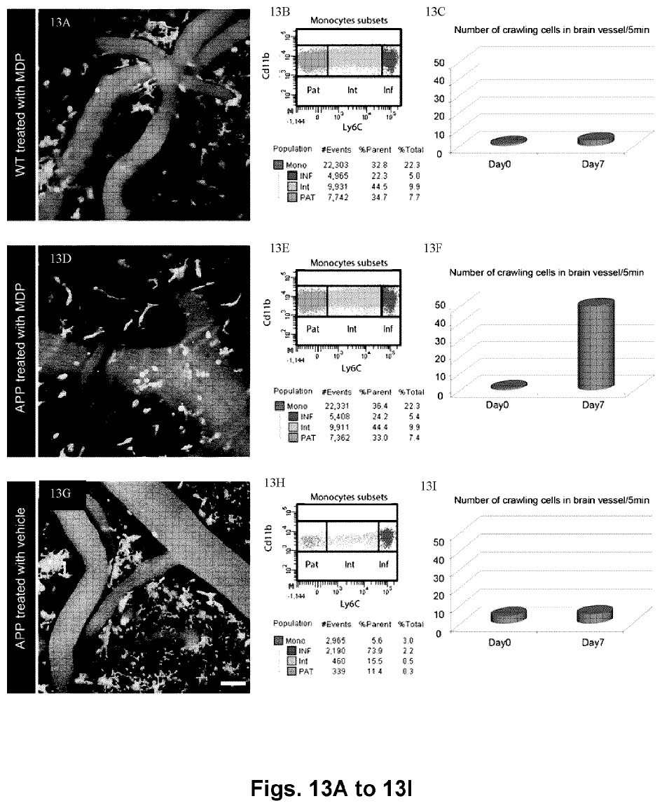

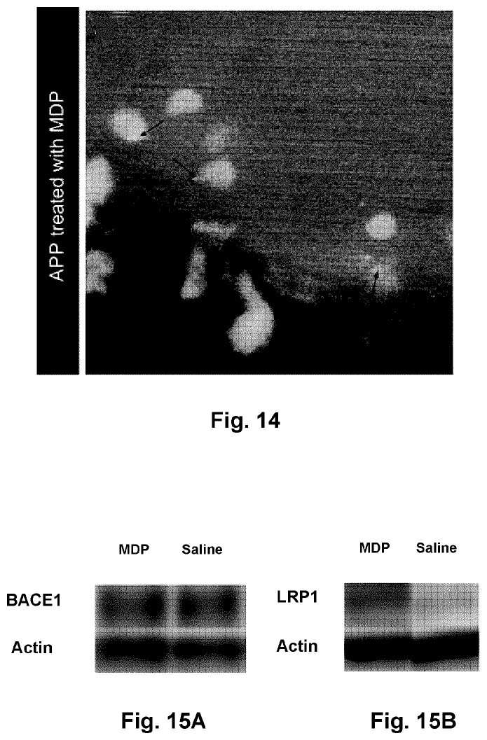

The results so far showed that MDP did not modulate microglial response in the brain, however the modulation of chemo-attractant factors, such as MCP1, can modulate monocyte recruitment to the brain. Therefore, the inventors performed live intravital two-photon microscopy in 12 month-old triple-transgenic APP_{swe}/PS1^{+/-}/Cx3CR1^{gfp/+} mice or in Cx3CR1^{gfp/+} mice. In this model, CX3CR1^{gfp/+}-expressing cells such as microglia, perivascular macrophages, and monocytes are green. Mice were injected with either MDP or saline for four consecutive days. It was first observed an increase in the percentage of patrolling monocytes (Ly6C^{low} monocytes) in WT (FIG. 13B) and APP (FIG. 13E) mice treated with MDP compared to saline controls (FIG. 13H). Next, a 5-minute time lapse quantification of CX3CR1^{gfp/+}-expressing cells in cortical blood vessels was performed 1 week after the first injection of MDP. It was then observed that crawling GFP⁺-cells were significantly more frequent in vessels containing small Aβ aggregates in APP mice treated with MDP compared to APP mice treated with saline (FIGS. 13D, 13F, 13G, and 13I). In addition, no significant crawling GFP⁺ cells were observed in WT mice treated with MDP (FIGS. 13A and 13C). More importantly, it was found that these crawling patrolling monocytes are selectively attracted to small Aβ aggregates present on APP cortical blood vessels (FIG. 14A). Together, these results confirm that MDP drives monocyte modulation towards Ly6C^{low} patrolling monocytes, which are selectively attracted to small Aβ aggregates to potentially mediate vascular Aβ clearance.

### Example X

### MDP Treatment Increases the Levels of LRP1 Receptors in the Brain of APP Mice

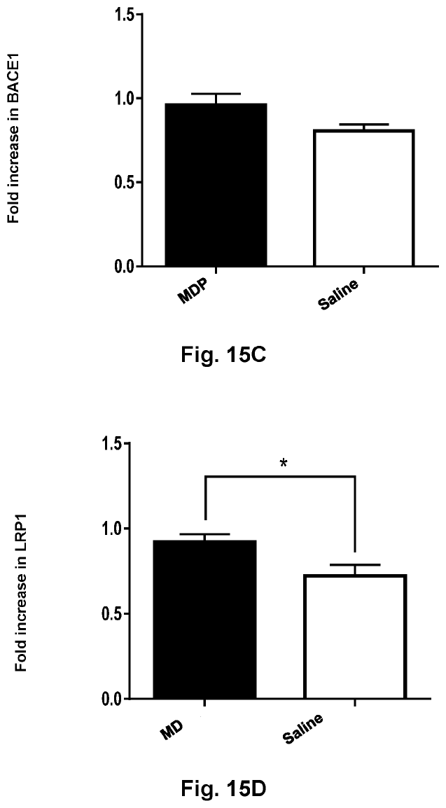

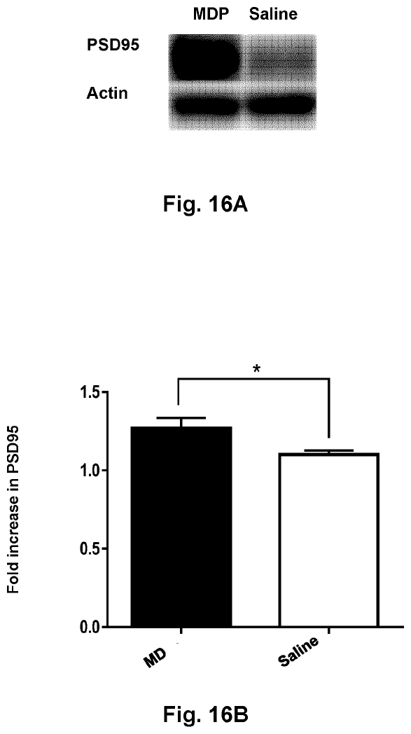

Low density lipoprotein receptor-related protein 1 (LRP1) is expressed in the neurovascular unit (NVU) and plays a critical role in the transport of Aβ from the abluminal to the luminal side of the BBB. Such a sink mechanism is involved in the clearance of Aβ from the brain parenchyma to the brain microvasculature, which is a direct target of circulating patrolling monocytes. It is interesting to note the significant higher levels of LRP1 in the brain of APP mice following MDP treatment (FIGS. 15B and 15D), whereas BACE1 remained unchanged (FIGS. 15A and 15C). This suggests that the NOD2 agonist acts mainly on the clearance and not on the synthesis facet of Aβ. Indeed, BACE1 plays a critical in the production of Aβ by neurons via the cleavage of APP. MDP does not seem to affect this process in the brain of APP mice and consequently does not seem to be involved in the neuroprotective properties of the drug. Although the biosynthesis of Aβ is not affected in response to the NOD2 agonist, elimination of Aβ from the brain via LRP1 transport across the BBB (abluminal to the luminal side) is significantly improved in presence of MDP.

### Example XI

### PSD95 is Significantly Increased in the Brain of APP Mice Following MDP Treatment

Synaptic activity and health are characteristics of memory function and specific proteins play a key role in modulating pre- and postsynaptic interactions. Decreased levels of these proteins in the postsynaptic fence evaluated here by Western blot on brain lysates precede the memory decline and neurodegeneration in APP/PS1 mice. Here, postsynaptic density protein 95 (PSD-95) was used as a marker of such postsynaptic activity in the brain of both MDP-treated and control APP mice. PSD-95 level is also increased in response to MDP administrations indicating improvement of post-synaptic functions (FIGS. 16A and 16B). The ability of the treatment to increase PSD95 in the brain of APP mice correlates with the effect on the improved cognitive decline that was evaluated with neurobehavioral tests in 6 months old APP mice. These data suggest MDP treatment improves postsynaptic functions via the increased expression of PSD95 in postsynaptic fences.

### Example XII

### MDP Treatment Significantly Increases the Level of COX2 and MCP1 in the Brain of APP Mice

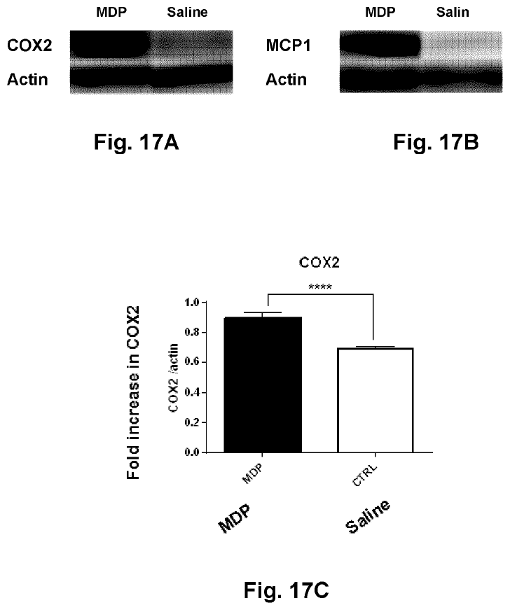

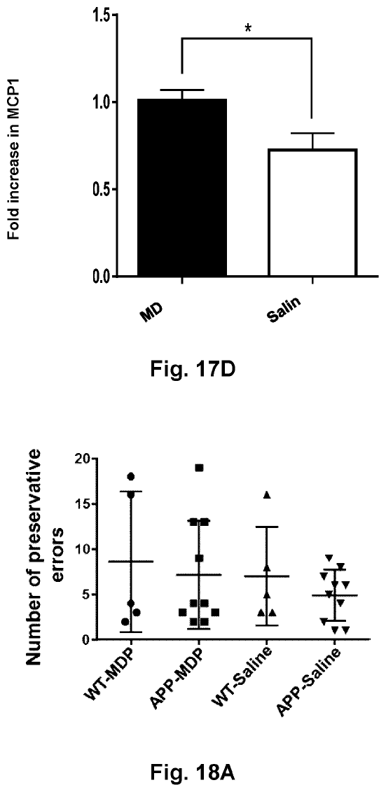

Patrolling monocytes are attracted to the luminal side of Aβ-containing blood vessels via the chemokine MCP-1, which is significantly increased by the NOD2 agonist MDP (FIGS. 17B and 17D). Together with the increased expression of COX-2 (FIGS. 17A and 17C), these data indicate that MDP triggers the brain Aβ clearance in converting inflammatory monocytes into patrolling cells and attracting them to the luminal side of Aβ-containing vascular elements to clear the toxic protein via phagocytosis. The inflammatory process in cells of the neurovascular unit is needed to attract patrolling cells at the luminal side of the BBB. Aβ is a direct trigger of this process since there are no crawling monocytes in the brain of intact wild-type mice. Combining both Aβ and MDP further stimulated such inflammatory processes that are absolutely needed for attracting patrolling monocytes into the luminal side of the BBB.

### Example XIII

### MDP Treatment Reduces the Number of Regressive Errors in APP Mice

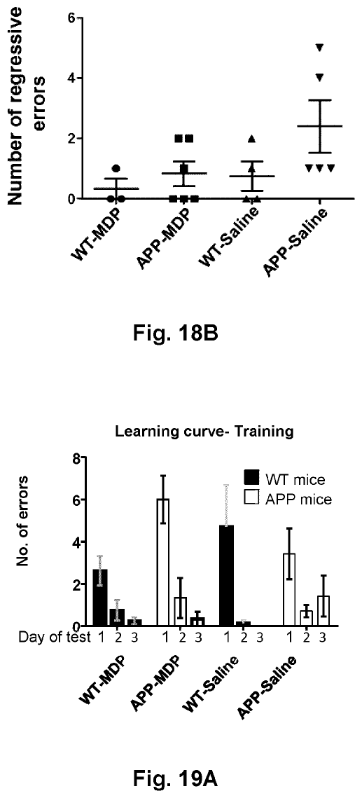

An ultimate consequence of these changes is the improved cognitive impairment associated with the disease. To evaluate these behavioral outcomes, mice were exposed to a series of tests, such as the T-water maze paradigm, a left/right discrimination test that assesses the hippocampal-based learning and retention of mice. The test was performed to measure cognitive functions and deficits. An escape platform is placed at the end of the target arm and is submerged 1 cm below the surface. In the acquisition-learning phase, mice are placed in the stem of the T-maze and swim freely until they find the submerged platform (located either in the right or in the left arm of the T-maze apparatus) and escape to it. The reversal-learning phase is then conducted 2 days later, with the protocol repeated except that the mice were trained to find the escape platform on the opposite side. The number of errors is indicative of cognitive decline. Higher number of regressive errors provides direct evidence of more cognitive impairment in a group of APP mice. In this regard, the number of regressive errors were lower in APP mice treated with MDP than those that received the saline solution, indicating an improved cognitive impairment in the group that was treated with the NOD2 agonist (FIGS. 18A and 18B). It is interesting to note that the number of regressive errors is actually similar to those of wild-type animals suggesting a normalization of the cognitive functions in APP mice treated with MDP.

### Example XIV

### MDP Treatment Reduces the Number of Reversal Errors in APP Mice

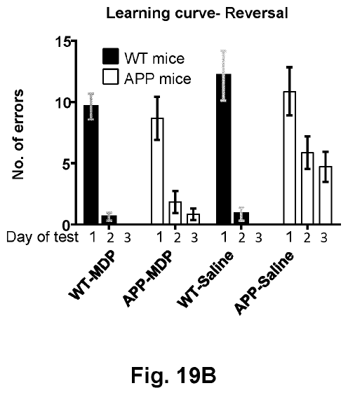

The T-water maze paradigm, the number of errors by trial to reach the criterion, and the average of swimming speeds have also been recorded and analyzed. The first 3 trials, represented in FIGS. 19A and 19B, demonstrate a significant improvement in the trials 2 and 3 after the MDP treatment, compared to APP mice treated with the saline solution. The number of errors in the trial 1 is similar for all the groups since they have to learn the novel task of the platform on the opposite side during the reversal phase of the test. The trials 2 and 3 are therefore quite important to discriminate the ability of the mice to learn a novel task, which is the reversal phase. MDP-treated APP mice made less errors in these two trials compared to mice that were treated with the control solution, which reinforced the previous behavioral data that NOD2 stimulation ameliorates the cognitive functions in this mouse model of AD.

These data together indicate that the improved cognitive functions of APP mice in presence of the NOD2 agonist may be dependent on the mechanism involved in the Aβ clearance from the BBB luminal side by the ability of patrolling monocytes to phagocyte the toxic protein. Having less Aβ may favor cognitive improvement and a less toxic environment for synaptic activity and health preventing consequently neurodegeneration.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art, in light of the above disclosure. The scope of the invention is to be defined only by the claims appended hereto, and by their equivalents.

### REFERENCES

Agrawal, S. M., J. Williamson, R. Sharma, H. Kebir, K. Patel, A. Prat and V. W. Yong (2013). “Extracellular matrix metalloproteinase inducer shows active perivascular cuffs in multiple sclerosis.” Brain 136: 1760-1777.
Annibali, V., G. Ristori, D. F. Angelini, B. Serafini, R. Mechelli, S. Cannoni, S. Romano, A. Paolillo, H. Abderrahim and A. Diamantini (2011). “CD161highCD8+ T cells bear pathogenetic potential in multiple sclerosis.” Brain 134: 542-554.
Bauer, J., I. Huitinga, W. Zhao, H. Lassmann, W. F. Hickey and C. D. Dijkstra (1995). “The role of macrophages, perivascular cells, and microglial cells in the pathogenesis of experimental autoimmune encephalomyelitis.” Glia 15: 437-446.
Bell, R. D., A. P. Sagare, A. E. Friedman, G. S. Bedi, D. M. Holtzman, R. Deane and B. V. Zlokovic (2007). “Transport pathways for clearance of human Alzheimer's amyloid β-peptide and apolipoproteins E and J in the mouse central nervous system.” Journal of Cerebral Blood Flow & Metabolism 27: 909-918.
Bell R D, A P Sagare, A E Friedman, G S Bedi, D M Holtzman, R Deane, and B V Zlokovic (2007). Transport pathways for clearance of human Alzheimer's amyloid β-peptide and apolipoproteins E and J in the mouse central nervous system. J Cereb Blood Flow Metab 27: 909-918.
Benveniste, E. N. (1997). “Role of macrophages/microglia in multiple sclerosis and experimental allergic encephalomyelitis.” Journal of Molecular Medicine 75(3): 165-173.
Bosetti, F., R. Langenbach and G. R. Weerasinghe (2004). “Prostaglandin E2 and microsomal prostaglandin E synthase-2 expression are decreased in the cyclooxygenase-2-deficient mouse brain despite compensatory induction of cyclooxygenase-1 and Ca2+-dependent phospholipase A2.” Journal of Neurochemistry 91: 1389-1397.
Bragg, D., J. Boles and R. Meeker (2002). “Destabilization of neuronal calcium homeostasis by factors secreted from choroid plexus macrophage cultures in response to feline immunodeficiency virus.” Neurobiology of Disease 9: 173-186.
Bragg, D., L. Hudson, Y. Liang, M. Tompkins, A. Fernandes and R. Meeker (2002). “Choroid plexus macrophages proliferate and release toxic factors in response to feline immunodeficiency virus.” Journal of Neurovirology 8: 225-239.
Brown, D. A. and P. E. Sawchenko (2007). “Time course and distribution of inflammatory and neurodegenerative events suggest structural bases for the pathogenesis of experimental autoimmune encephalomyelitis.” Journal of Comparative Neurology 502: 236-260.
Brunet, A., M. LeBel, B. Egarnes, C. Paquet-Bouchard, A. J. Lessard, J. P. Brown and J. Gosselin (2016). “NR4A1-dependent Ly6C^{low} monocytes contribute to reducing joint inflammation in arthritic mice through Treg cells.” European Journal of Immunology 46: 2789-2800.
Bustos, F. J., E. Ampuero, N. Jury, R. Aguilar, F. Falahi, J. Toledo, J. Ahumada, J. Lata, P. Cubillos and B. Henriquez (2017). “Epigenetic editing of the Dlg4/PSD95 gene improves cognition in aged and Alzheimer's disease mice.” Brain 140: 3252-3268.
Cao, D., V. Malmstrom, C. Baecher-Allan, D. Hafler, L. Klareskog and C. Trollmo (2003). “Isolation and functional characterization of regulatory CD25brightCD4+ T cells from the target organ of patients with rheumatoid arthritis.” European Journal of Immunology 33: 215-223.
Chakraborty, A., De Wit, N., Van Der Flier, W., and De Vries, H. (2017). The blood brain barrier in Alzheimer's disease. Vascular pharmacology 89, 12-18.
Deane, R., A. Sagare, K. Hamm, M. Parisi, S. Lane, M. B. Finn, D. M. Holtzman and B. V. Zlokovic (2008). “apoE isoformspecific disruption of amyloid β peptide clearance from mouse brain.” The Journal of Clinical Investigation 118: 4002-4013.
DeKosky, S. T. and S. W. Scheff (1990). “Synapse loss in frontal cortex biopsies in Alzheimer's disease: correlation with cognitive severity.” Annals of Neurology 27: 457-464.
Döring, A., S. Sloka, L. Lau, M. Mishra, J. van Minnen, X. Zhang, D. Kinniburgh, S. Rivest and V. W. Yong (2015). “Stimulation of monocytes, macrophages, and microglia by amphotericin B and macrophage colony-stimulating factor promotes remyelination.” Journal of Neuroscience 35: 1136-1148.
Elias, G. M. and R. A. Nicoll (2007). “Synaptic trafficking of glutamate receptors by MAGUK scaffolding proteins.” Trends in Cell Biology 17: 343-352.
Engelhardt, B., K. Wolburg-Buchholz and H. Wolburg (2001). “Involvement of the choroid plexus in central nervous system inflammation.” Microscopy Research and Technique 52: 112-129.
Feinen B. et al., Clin. Vaccine Immunol. 21: 580-586, 2014.
Fiala et al. J Alzheimers Dis. 7: 221-232, 2005.
Fife, B. T., G. B. Huffnagle, W. A. Kuziel and W. J. Karpus (2000). “CC chemokine receptor 2 is critical for induction of experimental autoimmune encephalomyelitis.” Journal of Experimental Medicine 192: 899-906.
Fritz J. H. et al., Nature Immunol 7: 1250-1257, 2006.
Fritz J. H. et al., Eur. J. Immunol. 35: 2459-2470, 2005.
Fujimura T. et al., J. Dermatol. 62: 107-115, 2011.
Girardin et al. J. Biol. Chem. 278: 41702-41708, 2003.
Girardin, S. E., I. G. Boneca, J. Viala, M. Chamaillard, A. Labigne, G. Thomas, D. J. Philpott and P. J. Sansonetti (2003). “Nod2 is a general sensor of peptidoglycan through muramyl dipeptide (MDP) detection.” Journal of Biological Chemistry 278: 8869-8872.
Greter, M., F. L. Heppner, M. P. Lemos, B. M. Odermatt, N. Goebels, T. Laufer, R. J. Noelle and B. Becher (2005). “Dendritic cells permit immune invasion of the CNS in an animal model of multiple sclerosis.” Nature Medicine 11: 328-334.
Guariglia, S. R. and K. K. Chadman (2013). “Water T-maze: a useful assay for determination of repetitive behaviors in mice.” Journal of Neuroscience Methods 220: 24-29.
Hong, S., V. F. Beja-Glasser, B. M. Nfonoyim, A. Frouin, S. Li, S. Ramakrishnan, K. M. Merry, Q. Shi, A. Rosenthal and B. A. Barres (2016). “Complement and microglia mediate early synapse loss in Alzheimer mouse models.” Science 352: 712-716.
Hou, Y., M. A. Aboukhatwa, D.-L. Lei, K. Manaye, I. Khan and Y. Luo (2010). “Anti-depressant natural flavonols modulate BDNF and beta amyloid in neurons and hippocampus of double TgAD mice.” Neuropharmacology 58: 911-920.
Huber, M., S. Heink, A. Pagenstecher, K. Reinhard, J. Ritter, A. Visekruna, A. Guralnik, N. Bollig, K. Jeltsch and C. Heinemann (2012). “IL-17A secretion by CD8+ T cells supports Th17-mediated autoimmune encephalomyelitis.” The Journal of Clinical Investigation 123: 247-260.
Hui, C. W., M.-K. St-Pierre, J. Detuncq, L. Aumailley, M.-J. Dubois, V. Couture, D. Skuk, A. Marette, J. P. Tremblay and M. Lebel (2018). “Nonfunctional mutant Wrn protein leads to neurological deficits, neuronal stress, microglial alteration, and immune imbalance in a mouse model of Werner syndrome.” Brain, Behavior, and Immunity 73: 450-469.
Huppert, J., D. Closhen, A. Croxford, R. White, P. Kulig, E. Pietrowski, I. Bechmann, B. Becher, H. J. Luhmann and A. Waisman (2010). “Cellular mechanisms of IL-17-induced blood-brain barrier disruption.” The FASEB Journal 24: 1023-1034.
Iliff, J. J., Wang, M., Liao, Y., Plogg, B. A., Peng, W., Gundersen, G. A., et al. (2012). A paravascular pathway facilitates CSF flow through the brain parenchyma and the clearance of interstitial solutes, including amyloid β. Science translational medicine 4(147), 147ra111-147ra111.
Inohara N. et al., J. Biol. Chem. 278: 5509-5512, 2013.
Izikson, L., R. S. Klein, A. D. Luster and H. L. Weiner (2002). “Targeting monocyte recruitment in CNS autoimmune disease.” Clinical Immunology 103: 125-131.
Kanekiyo, T., J. R. Cirrito, C.-C. Liu, M. Shinohara, J. Li, D. R. Schuler, M. Shinohara, D. M. Holtzman and G. Bu (2013). “Neuronal clearance of amyloid-β by endocytic receptor LRP1.” Journal of Neuroscience 33: 19276-19283.
Kanekiyo, T., C.-C. Liu, M. Shinohara, J. Li and G. Bu (2012). “LRP1 in brain vascular smooth muscle cells mediates local clearance of Alzheimer's amyloid-β.” Journal of Neuroscience 32: 16458-16465.
Kang, D. E., C. U. Pietrzik, L. Baum, N. Chevallier, D. E. Merriam, M. Z. Kounnas, S. L. Wagner, J. C. Troncoso, C. H. Kawas and R. Katzman (2000). “Modulation of amyloid β-protein clearance and Alzheimer's disease susceptibility by the LDL receptor-related protein pathway.” The Journal of Clinical Investigation 106: 1159-1166.
King, I. L., T. L. Dickendesher and B. M. Segal (2009). “Circulating Ly-6C+ myeloid precursors migrate to the CNS and play a pathogenic role during autoimmune demyelinating disease.” Blood 113: 3190-3197.
Laflamme, N., G. Cisbani, P. Préfontaine, Y. Srour, J. Bernier, M.-K. St-Pierre, M.-È. Tremblay and S. Rivest (2018). “mCSF-induced microglial activation prevents myelin loss and promotes its repair in a mouse model of multiple sclerosis.” Frontiers in Cellular Neuroscience 12: 178. doi.org/10.3389/fncel.2018.00178
Laflamme, N., S. Lacroix and S. Rivest (1999). “An essential role of interleukin-1β in mediating NF-κB activity and COX-2 transcription in cells of the blood-brain barrier in response to a systemic and localized inflammation but not during endotoxemia.” Journal of Neuroscience 19: 10923-10930.
Laflamme, N. and S. Rivest (2001). “Toll-like receptor 4: the missing link of the cerebral innate immune response triggered by circulating gram-negative bacterial cell wall components.” The FASEB Journal 15: 155-163.
Lampron, A., A. Larochelle, N. Laflamme, P. Préfontaine, M.-M. Plante, M. G. Sánchez, V. W. Yong, P. K. Stys, M.-È. Tremblay and S. Rivest (2015). “Inefficient clearance of myelin debris by microglia impairs remyelinating processes.” Journal of Experimental Medicine 212: 481-495.
Lannfelt L. et al., J. Int. Med. 275: 284-295, 2014
Lee, S. Y. and J. M. Goverman (2013). “The influence of T cell Ig mucin-3 signaling on central nervous system autoimmune disease is determined by the effector function of the pathogenic T cells.” The Journal of Immunology 190: 4991-4999.
Lessard, A.-J., M. LeBel, B. Egarnes, P. Préfontaine, P. Thériault, A. Droit, A. Brunet, S. Rivest and J. Gosselin (2017). “Triggering of NOD2 Receptor Converts Inflammatory Ly6C high into Ly6C low Monocytes with Patrolling Properties.” Cell Reports 20: 1830-1843.
Liu, L., and Duff, K. (2008). A technique for serial collection of cerebrospinal fluid from the cisterna magna in mouse. JoVE (Journal of Visualized Experiments) (21), e960.
Luger, D., P. B. Silver, J. Tang, D. Cua, Z. Chen, Y. Iwakura, E. P. Bowman, N. M. Sgambellone, C.-C. Chan and R. R. Caspi (2008). “Either a Th17 or a Th1 effector response can drive autoimmunity: conditions of disease induction affect dominant effector category.” Journal of Experimental Medicine 205: 799-810.
Martin, A. M., C. Kuhlmann, S. Trossbach, S. Jaeger, E. Waldron, A. Roebroek, H. J. Luhmann, A. Laatsch, S. Weggen and V. Lessmann (2008). “The functional role of the second NPXY motif of the LRP1 β-chain in tissue-type plasminogen activator-mediated activation of N-methyl-D-aspartate receptors.” Journal of Biological Chemistry 283: 12004-12013.
Michaud, J.-P., M.-A. Bellavance, P. Préfontaine and S. Rivest (2013). “Real-time in vivo imaging reveals the ability of monocytes to clear vascular amyloid beta.” Cell Reports 5: 646-653.
Michaud, J.-P., M. Hallé, A. Lampron, P. Thériault, P. Préfontaine, M. Filali, P. Tribout-Jover, A.-M. Lanteigne, R. Jodoin and C. Cluff (2013). “Toll-like receptor 4 stimulation with the detoxified ligand monophosphoryl lipid A improves Alzheimer's disease-related pathology.” Proceedings of the National Academy of Sciences of the U.S.A. 110: 1941-1946.
Minghetti, L. (2004). “Cyclooxygenase-2 (COX-2) in inflammatory and degenerative brain diseases.” Journal of Neuropathology & Experimental Neurology 63: 901-910.
Mishra, M. K., J. Wang, C. Silva, M. Mack and V. W. Yong (2012). “Kinetics of proinflammatory monocytes in a model of multiple sclerosis and its perturbation by laquinimod.” The American Journal of Pathology 181: 642-651.
Moliné-Velázquez, V., H. Cuervo, V. Vila-del Sol, M. C. Ortega, D. Clemente and F. de Castro (2011). “Myeloid-derived suppressor cells limit the inflammation by promoting T lymphocyte apoptosis in the spinal cord of a murine model of multiple sclerosis.” Brain Pathology 21: 678-691.
Mossakowski, A. A., J. Pohlan, D. Bremer, R. Lindquist, J. M. Millward, M. Bock, K. Pollok, R. Mothes, L. Viohl and M. Radbruch (2015). “Tracking CNS and systemic sources of oxidative stress during the course of chronic neuroinflammation.” Acta Neuropathologica 130: 799-814.
Mostany, R., and Portera-Cailliau, C. (2008). A craniotomy surgery procedure for chronic brain imaging. JoVE (Journal of Visualized Experiments) (12), e680.
Nazer, B., S. Hong and D. J. Selkoe (2008). “LRP promotes endocytosis and degradation, but not transcytosis, of the amyloid-β peptide in a blood-brain barrier in vitro model.” Neurobiology of Disease 30: 94-102.
Niethammer, M., E. Kim and M. Sheng (1996). “Interaction between the C terminus of NMDA receptor subunits and multiple members of the PSD-95 family of membrane-associated guanylate kinases.” Journal of Neuroscience 16: 2157-2163.
Nikić, I., D. Merkler, C. Sorbara, M. Brinkoetter, M. Kreutzfeldt, F. M. Bareyre, W. Brack, D. Bishop, T. Misgeld and M. Kerschensteiner (2011). “A reversible form of axon damage in experimental autoimmune encephalomyelitis and multiple sclerosis.” Nature Medicine 17: 495-499.
Nygaard H. B., Clin. Ther. 35:1480-1489, 2013.
O'Connor, R. A., K. H. Malpass and S. M. Anderton (2007). “The inflamed central nervous system drives the activation and rapid proliferation of Foxp3+ regulatory T cells.” The Journal of Immunology 179: 958-966.
Rangachari, M. and V. K. Kuchroo (2013). “Using EAE to better understand principles of immune function and autoimmune pathology.” Journal of Autoimmunity 45: 31-39.
Sang, N. and C. Chen (2006). “Lipid signaling and synaptic plasticity.” The Neuroscientist 12: 425-434.
Silverberg, G. D., A. A. Messier, M. C. Miller, J. T. Machan, S. S. Majmudar, E. G. Stopa, J. E. Donahue and C. E. Johanson (2010). “Amyloid efflux transporter expression at the blood-brain barrier declines in normal aging.” Journal of Neuropathology & Experimental Neurology 69: 1034-1043.
Simard, A. R., Soulet, D., Gowing, G., Julien, J.-P., and Rivest, S. (2006). Bone marrow-derived microglia play a critical role in restricting senile plaque formation in Alzheimer's disease. Neuron 49(4), 489-502.
Sloka, S., L. M. Metz, W. Hader, Y. Starreveld and V. W. Yong (2013). “Reduction of microglial activity in a model of multiple sclerosis by dipyridamole.” Journal of Neuroinflammation 10: 855.
Sorokin, L. (2010). “The impact of the extracellular matrix on inflammation.” Nature Reviews Immunology 10: 712-723.
Storck, S. E., S. Meister, J. Nahrath, J. N. MeiBner, N. Schubert, A. Di Spiezio, S. Baches, R. E. Vandenbroucke, Y. Bouter and I. Prikulis (2016). “Endothelial LRP1 transports amyloid-β 1-42 across the blood-brain barrier.” The Journal of Clinical Investigation 126: 123-136.
Terry, R. D., E. Masliah, D. P. Salmon, N. Butters, R. DeTeresa, R. Hill, L. A. Hansen and R. Katzman (1991). “Physical basis of cognitive alterations in Alzheimer's disease: synapse loss is the major correlate of cognitive impairment.” Annals of Neurology 30: 572-580.
Thériault, P., A. ElAli and S. Rivest (2016). “High fat diet exacerbates Alzheimer's disease-related pathology in APPswe/PS1 mice.” Oncotarget 7: 67808-67827.
Tran, E. H., K. Hoekstra, N. van Rooijen, C. D. Dijkstra and T. Owens (1998). “Immune invasion of the central nervous system parenchyma and experimental allergic encephalomyelitis, but not leukocyte extravasation from blood, are prevented in macrophage-depleted mice.” The Journal of Immunology 161: 3767-3775.
Van Horssen, J., M. E. Witte, G. Schreibelt and H. E. De Vries (2011). “Radical changes in multiple sclerosis pathogenesis.” Biochimica et Biophysica Acta (BBA)-Molecular Basis of Disease 1812: 141-150.
Van Zundert, B., A. Yoshii and M. Constantine-Paton (2004). “Receptor compartmentalization and trafficking at glutamate synapses: a developmental proposal.” Trends in Neurosciences 27: 428-437.
Vercellino, M., B. Votta, C. Condello, C. Piacentino, A. Romagnolo, A. Merola, E. Capello, G. L. Mancardi, R. Mutani and M. T. Giordana (2008). “Involvement of the choroid plexus in multiple sclerosis autoimmune inflammation: a neuropathological study.” Journal of Neuroimmunology 199: 133-141.
Vernet-der Garabedian, B., Y. Lemaigre-Dubreuil and J. Mariani (2000). “Central origin of IL-1β produced during peripheral inflammation: role of meninges.” Molecular Brain Research 75: 259-263.
Yamasaki, R., H. Lu, O. Butovsky, N. Ohno, A. M. Rietsch, R. Cialic, P. M. Wu, C. E. Doykan, J. Lin and A. C. Cotleur (2014). “Differential roles of microglia and monocytes in the inflamed central nervous system.” Journal of Experimental Medicine 211: 1533-1549.
Yang, H. and C. Chen (2008). “Cyclooxygenase-2 in synaptic signaling.” Current Pharmaceutical Design 14: 1443-1451.
Zhao, Z., A. P. Sagare, Q. Ma, M. R. Halliday, P. Kong, K. Kisler, E. A. Winkler, A. Ramanathan, T. Kanekiyo and G. Bu (2015). “Central role for PICALM in amyloid-β blood-brain barrier transcytosis and clearance.” Nature Neuroscience 18: 978-987.

1-6. (canceled)

7. A method for reducing A8 in a patient, said method comprising the step of administering to a patient in need thereof a therapeutically effective dose of a NOD2 agonist.

8. The method of claim 7, wherein transport of A8 across the blood brain barrier is increased.

9. The method of claim 7, wherein production of Low density lipoprotein receptor-related protein 1 (LRP1) is increased, causing an increase of transport of A8 across the blood brain barrier.

10. A method for treating a patient afflicted with Alzheimer's disease (AD) or multiple sclerosis (MS), said method comprising the step of administering to said patient a therapeutically effective dose of a NOD2 agonist.

11. A method for the improvement of cognitive disorder or learning and memory disorder associated with Alzheimer's disease (AD), said method comprising the step of administering to a patient in need thereof a therapeutically effective dose of a NOD2 agonist.

12. The method of claim 10, wherein production of postsynaptic density protein 95 (PSD-95) is increased.

13-30. (canceled)

31. The method of claim 11, wherein production of postsynaptic density protein 95 (PSD-95) is increased.
